(19) United States
(12) Patent Application Publication
(10) Pub. No.: US 2018/0245049 A1
(43) Pub. Date: Aug. 30, 2018

(54) METHOD FOR INDUCING DIFFERENTIATION OF STEM CELLS

(71) Applicant: STICHTING KATHOLIEKE UNIVERSITEIT, Nijmegen (NL)

(72) Inventors: Wouter Franciscus Joannes FEITZ, Nijmegen (NL); Egbert OOSTERWIJK, Nijmegen (NL); Silvia Maria MIHAILA, Nijmegen (NL); Alan Edward ROWAN, Nijmegen (NL)

(21) Appl. No.: 15/757,218

(22) PCT Filed: Sep. 5, 2016
(86) PCT No.: PCT/EP2016/070876

§ 371 Date: Mar. 2, 2018

(30) Foreign Application Priority Data
Sep. 3, 2015 (EP) 15183759.8
Sep. 9, 2015 (EP) 15184507.0
Sep. 9, 2015 (EP) 15184524.5
Sep. 9, 2015 (EP) 15184530.2
Nov. 27, 2015 (EP) 15196825.2
Nov. 27, 2015 (EP) 15196845.0
Nov. 27, 2015 (EP) 15196856.7

(51) Int. Cl. C12N 5/0775; C12N 5/00

(52) U.S. Cl. CPC C12N 5/0663; C12N 5/0062; C12N 2506/1346; C12N 2533/32; C12N 2527/00; C12N 2533/40; C12N 2513/00

(57) ABSTRACT

A cell culture including a cell culturing medium for growing stem cells, a three-dimensional (3D) cell growth matrix and stem cells, wherein the cell culture has a critical stress σ_{c} of 2-30 Pa, wherein the critical stress is a stress which marks an onset of strain stiffening and wherein the cell culture has a storage modulus G′ measured at 37° C. of 50-1000 Pa.

### FIELD OF THE INVENTION

The present invention relates to a method for inducing differentiation of stem cells and to a cell culture obtainable by this method.

### BACKGROUND OF THE INVENTION

There is a need for culturing of different specialized cells in modern regenerative medicine, for example for generating new tissue or generating material for drug testing and the like.

WO 2011/007012 (which is incorporated herein by reference) discloses a hydrogel comprising oligo(alkylene glycol) functionalized polyisocyanopeptides. The polyisocyanopeptides are prepared by functionalizing an isocyanopeptide with oligo-(alkylene glycol) side chains and subsequently polymerizing the oligo-alkylene glycol functionalized isocyanopeptides.

WO2015/007771 (which is incorporated herein by reference) describes the use of polyisocyanopeptides which are modified with cell adhesion factors like GRD or GRGDS to support growth of cells.

The control of the differentiation of the stem cells has been performed in purely biological systems and has been unsuccessful in synthetic systems. There is a need for controlling the differentiation of stem cells in a more efficient, reliable manner. In particular, it would be desirable to be able to induce osteogenic differentiation of stem cells in a reliable manner.

### SUMMARY OF THE INVENTION

It is an objective of the present invention to provide a cell culture for differentiation of stem cells.

The invention relates to a cell culture comprising:

(a) a cell culturing medium for growing stem cells,

(b) a three-dimensional (3D) cell growth matrix and

(c) stem cells,

wherein the cell culture has a critical stress σ_{c} of 2-30 Pa, wherein the critical stress is a stress which marks an onset of strain stiffening and

wherein the cell culture has a storage modulus G′ measured at 37° C. of 50-1000 Pa.

Preferably, the cyc ranges between 5 and 25 Pa, and G′ ranges between 70 and 400 Pa.

Different 3D cell growth matrices can be used in the present invention, as long as they provide the claimed critical stress σ_{c} and storage modulus G′ to the cell culture.

Examples of 3D cell growth matrices are Matrigel®, Puramatrix®, Raft® 3D, Insphero®, Bioactive 3D®, Cellusponge®, Optimaix® and GroCell-3D® scaffolds.

In a preferred embodiment, the 3D cell growth matrix is a hydrogel.

More preferably, the 3D cell growth matrix comprises an oligo(alkylene glycol) substituted co-polyisocyanopeptide (PIC).

Preferably, the concentration of the polyisocyanopeptide in the 3D cell growth matrix is 1-5 mg/ml.

Preferably, the average length of the polyisocyanopeptide is 250-680 nm as determined by AFM.

Preferably, the polyisocyanopeptide has a cell adhesion factor covalently bound to the polyisocyanopeptide and/or the cell culturing medium comprises fibrin. In case the polyisocyanopeptide has a cell adhesion factor covalently bound to the polyisocyanopeptide, preferably the average distance between the cell adhesion factors along the polyisocyanopeptide backbone is 10-50 nm.

In a preferred embodiment, the invention relates to a cell culture comprising:

a) a cell culturing medium for growing stem cells,

b) a three-dimensional (3D) cell growth matrix and

c) stem cells,

wherein the 3D cell growth matrix comprises an oligo(alkylene glycol) substituted co-polyisocyanopeptide (PIC) preferably modified with cell adhesion factors and wherein the average distance between the cell adhesion factors along the polyisocyanopeptide backbone is 10-50 nm;

wherein the cell culture has a critical stress σ_{c} of 2-30 Pa and wherein the critical stress is a stress which marks an onset of strain stiffening; and

wherein the cell culture has a strorage modulus G′ measured at 37° C. of 50-1000 Pa.

The invention also relates to a method for inducing differentiation of stem cells, comprising the steps of

a) mixing a cell culturing medium for differentiation with an oligo(alkylene glycol) substituted co-polyisocyanopeptide at a temperature between 0 and 18° C. to obtain a polymer solution;

b) mixing the polymer solution with stem cells at a temperature between 0 and 18° C. to obtain a cell culture solution;

c) allowing the cell culture solution to warm to a temperature between 30 and 38° C. to form a cell culture comprising a hydrogel and allow the stem cells to differentiate,

wherein the concentration of the polyisocyanopeptide in the polymer solution is 1-5 mg/ml,

wherein the average length of the polyisocyanopeptide is 250-680 nm as determined by AFM,

wherein the cell density of the stem cells in the cell culture solution is 0.3*10⁶-1*10⁶ cells/ml,

wherein the hydrogel has a critical stress σ_{c} of 2-30 Pa, wherein the critical stress is a stress which marks an onset of a strain stiffening,

wherein the hydrogel has a storage modulus G′ measured at 37° C. of 50-1000 Pa and wherein the polyisocyanopeptide has a cell adhesion factor covalently bound to the polyisocyanopeptide and/or wherein the cell culturing medium comprises fibrin, wherein when the polyisocyanopeptide has a cell adhesion factor covalently bound to the polyisocyanopeptide, the average distance between the cell adhesion factors along the polyisocyanopeptide backbone is 10-50 nm.

The present method has an advantage that not only cell growth (spreading and proliferation) of stem cells can be stimulated in the presence of the polymer solution, but also differentiation of stem cells can be induced depending on the cell culturing medium and physical characteristic of the polymer solution. This gives a good control of growth of cells. This method can be used to generate specific tissue suitable for treatment of individual patients, after retrieval of stem cells from said patients. The time to grow and differentiate the stem cells to useful tissue can be shortened, and it is expected that the success of implantation of new tissue and cure of patients can be accelerated.

The present inventors have discovered a new method for growth and/or differentiation of stem cells using a three dimensional synthetic hydrogel. The stem cells are present inside the three dimensional hydrogel according to the invention. This is in contrast with state of the art cell culture media, wherein the hydrogel is a two dimensional hydrogel and the stem cells are present on the surface of the hydrogel.

The fact that a synthetic hydrogel can be used for desired growth and/or differentiation of stem cells is highly advantageous. Although not wishing to be bound by any theory, it is believed that the hydrogel used in the present invention has mechanical properties similar to natural systems surrounding the stem cell and give similar stimulations to the stem cells for growth and/or differentiation. It was found that the three dimensional hydrogel undergoes a strain stiffening which influences the growth and differentiation of the stem cells surrounded by the hydrogel. The strain stiffening of the hydrogel is believed to be transported via mechano transduction to the stem cells.

Surprisingly, differentiation of stem cells can be induced in a controlled manner by controlling the critical stress σ_{c} of the hydrogel according to the method of the invention.

The hydrogel according to the invention exhibits a substantially linear stress response at a low stress. When the stress is increased beyond a critical stress σ_{c}, the hydrogel exhibits a non-linear stress response, i.e. becomes stiffer with increasing applied stress.

The critical stress σ_{c} and its determination method are described in detail e.g. in Broedersz, C. P. et al. Measurement of nonlinear rheology of cross-linked biopolymer gels. Soft Matter 6, 4120 (2010), Kouwer, P. H. J. et al. Responsive biomimetic networks from polyisocyanopeptide hydrogels. Nature 493, 651-655 (2013) and Jaspers, M. et al. Ultra-responsive soft matter from strain-stiffening hydrogels. Nat. Commun. 5, 5808 (2014), all incorporated herein by reference in full.

The present inventors have surprisingly found that the critical stress of the hydrogel controls whether the stem cell exhibits osteogenic differentiation, vascularization or adipogenic differentiation. It was found that stem cells exhibit different types of differentiation depending on the critical stress σ_{c} of the hydrogel even when the storage modulus of the hydrogel was the same. When the critical stress σ_{c} is low such as below 13 Pa (i.e. the hydrogel starts to exhibit strain-stiffening at a low stress level), the stem cells exhibit vascularization. Preferably the critical stress σ_{c} ranges between 7 and 12 Pa. When the critical stress is higher, the stem cells exhibit adipogenic or osteogenic differentiation. For adipogenic, the preferred critical stress σ_{c} ranges between 7 and 23, preferably between 8 and 20 Pa. For osteogenic differentiation the critical stress σ_{c} ranges preferably between 5-25 or 11-30 or 12-22 or 13-20 Pa.

It was further found that the storage modulus G′ of the hydrogel of 50-1000 Pa was necessary for the differentiation of stem cells. A storage modulus lower than 50 Pa leads to more proliferation of the cells rather than the differentiation of stem cells. Preferably, the storage modulus G′ of the hydrogel is 80-500 Pa, more preferably 200-400 Pa. These ranges are found to be optimal for the differentiation of stem cells. It is to be noted that such storage modulus G′ is much lower compared to known systems and such hydrogels are considered very soft in the field.

The critical stress of the hydrogel can be tuned by the concentration of the polymer solution and the molecular weight of the polyisocyanopeptide.

Preferably, the concentration of the polyisocyanopeptide in the polymer solution is 1-5 mg/ml. Below the concentration of 1 mg/ml, the hydrogel is too weak and it is difficult for the hydrogel to maintain its shape and support the growing cells. Above the concentration of 5 mg/ml, the hydrogel tends to be too stiff, whereby it is difficult for the stem cells in the hydrogel to move and differentiate. In an embodiment, the concentration of the polyisocyanopeptide of the polymer solution is 1.5-3 mg/ml.

The polymer solution used in the present invention has a thermo-responsive character, i.e. the polymer solution turns into a hydrogel at or above the gelling temperature and the hydrogel turns to liquid (the polymer solution) by cooling it to a temperature below the gelling temperature.

In step c) of the method of the present invention, the cell culture is allowed to reach the gelling temperature of the polymer solution, i.e. the polymer solution takes the form of a hydrogel at a temperature where the stem cells are allowed to differentiate.

The polyisocyanopeptide has a cell adhesion factor covalently bound to the polyisocyanopeptide or the cell culturing medium comprises fibrin. Preferably, the polyisocyanopeptide has a cell adhesion factor covalently bound to the polyisocyanopeptide. Preferably, the average distance between the cell adhesion factors along the polymer backbone is 10-50 nm. Preferably the average distance between the cell adhesion factors along the polymer backbone is 15-35 nm, more preferably between 20-30 nm. The cell adhesion factor is important for the transduction of the mechanical properties of the polyisocyanopeptide to the growing cells. When the distance between the cell adhesion factors is shorter than 10 nm, the cells are hampered in growing and differentiation. When the distance between is too large (larger than 50 nm), or the cell adhesion factor is not present, and also no fibrin is present, than the cells do not experience any mechanotransduction, and no differentiation of cells will occur.

The cell density of the stem cells in the cell culture solution is 0.3*10⁶-1*10⁶ cells/ml. When the cell density in the cell culture solution is below 0.3*10⁶ cells/ml there are not enough stem cells present in the hydrogel to effectively grow and/or differentiate the stem cells. When the polymer concentration in the polymer solution is above 1*10⁶ cells/ml, there are too many stem cells present in the hydrogel causing crowding and exhaustion of the hydrogel. This slows down the growth and/or differentiation of the stem cells.

Polymer

Oligo(alkyleneglycol)-substituted polyisocyanopeptides which are being used in the context of the present invention can be described with the following formula

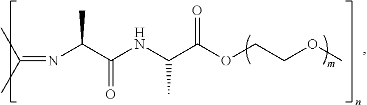

wherein m is an integer between 1 and 10, and wherein n is an integer between 1-100000.

An example of a methoxy-mono-ethyleneglycol substituted isocyanopeptide unit is:

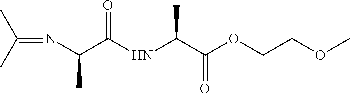 [SMILES: COCCOC(=O)[C@H](C)NC(=O)[C@@H](C)N=C(C)C]

An example of methoxy tetra-ethyleneglycol substituted isocyanopeptide unit is:

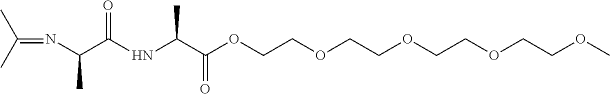 [SMILES: COCCOCCOCCOCCOC(=O)[C@H](C)NC(=O)[C@@H](C)N=C(C)C]

Such polymer is used either in combination with fibrin, and/or is substituted with one or more cell adhesion factors.

Fibrin

In an embodiment of the invention the oligo(alkylenglycol)-substituted polyisocyanopeptide is used in combination with fibrin. The inventors found that a combination of fibrin with an oligo(alkylenglycol)-substituted polyisocyanopeptide can generate a system that is viable for inducing differentiation of stem cells.

Preferably the weight ratio of fibrin to the polyisocyanopeptide ranges between 5:95 and 99.5:0.5. More preferably the ratio is between 10:90 and 75:25, between 15:85 and 50:50, between 20:80 and 40:60 or between 25:75 and 35:65.

Coupled Cell Adhesion Factor

In an embodiment the oligo(alkyleneglycol)-substituted copolyisocyanopeptide is obtained by copolymerizing a first comonomer of an oligo(alkylene glycol) substituted isocyanopeptide grafted with a cell adhesion factor and a second comonomer of a non-grafted oligo(alkylene glycol) substituted isocyanopeptide. It is noted that oligo(alkyleneglycol)-substituted copolyisocyanopeptide may also be referred as oligo(alkyleneglycol)-functionalized copolyisocyanopeptide.

According to one aspect, the oligo(alkylene glycol) substituted co-polyisocyanopeptide can be prepared by a process comprising the steps of:
i) copolymerizing
a first comonomer of an oligo(alkylene glycol) substituted isocyanopeptide grafted with a linking group and
a second comonomer of a non-grafted oligo(alkylene glycol) substituted isocyanopeptide,
wherein the molar ratio between the first comonomer and the second comonomer is 1:500 and 1:30 and
ii) adding a reactant of a spacer unit and a cell adhesion factor to the copolymer obtained by step i), wherein the spacer unit is represented by general formula A-L-B,
wherein the linking group and group A are chosen to react and form a first coupling and the cell adhesion factor and group B are chosen to react and form a second coupling,
wherein the first coupling and the second coupling are independently selected from the group consisting of alkyne-azide coupling, dibenzocyclooctyne-azide coupling, oxanorbornadiene-based-azide couplings, vinylsulphone-thiol coupling, maleimide-thiol coupling, methyl methacrylate-thiol coupling, ether coupling, thioether coupling, biotin-strepavidin coupling, amine-carboxylic acid resulting in amides linkages, alcohol-carboxylic acid coupling resulting in esters linkages and NHS-Ester (N-Hydroxysuccinimide ester)-amine coupling and
wherein group L is a linear chain segment having 10-60 bonds between atoms selected from C, N, O and S in the main chain.

The linking group and group A are chosen to react and form a first coupling which may be any coupling mentioned in the above list. For example, in order to obtain an alkyne-azide coupling, the linking group may be alkyne and group A may be azide or the linking group may be azide and group A may be alkyne. The couplings mentioned in the above list are well-known to the skilled person and the formation of the couplings are found in textbooks. For example, NH₂—COOH coupling can be mediated via EDC.

Preferably, the first coupling is an alkyne-azide coupling.

Similarly, the cell adhesion factor and group B are chosen to react and form a second coupling which may be any coupling mentioned in the above list. Preferably, the second coupling is NHS-Ester (N-Hydroxysuccinimide ester)-amine coupling or maleimide-thiol coupling. This may be a coupling of NHS-ester to the N terminus of a the cell adhesion factor being a peptide or a coupling of maleimide to a terminal thiol of the cell adhesion factor being a peptide.

Group L is a segment having a linear chain connecting reactive groups A and B. The segment is formed by a sequence of atoms selected from C, N, O and S. The number of bonds between the atoms in the main chain connected to groups A and B is at least 10 and at most 60. The term ‘main chain’ is understood to mean the chain which connects the groups A and B with the shortest distance. The number of bonds between the atoms in the main chain connected to the terminal groups A and B is preferably at least 12, more preferably at least 15. The number of bonds between the atoms in the main chain connected to the terminal groups A and B is preferably at least 50, more preferably at least 40.

It was found that a certain minimum distance between the copolymer backbone and the cell adhesion factor is required for the cells attached to the cell adhesion factor to be cultured. The distance given by at least 10 bonds was found to be necessary, which is provided by the presence of the spacer unit according to the invention. The length below 10 bonds was found not to allow sufficient cell growth.

Preferred examples of group L are the following:

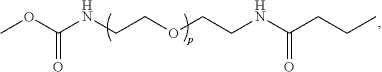

where p is 1 to 10, preferably 2 to 5,

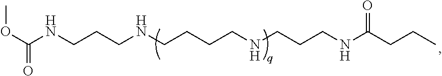

where q is 1 to 9, preferably 2 to 5,

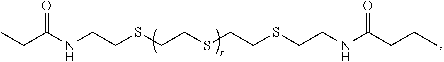

where r is 1 to 10, preferably 2 to 5.

When the spacer unit contains these types of group L, particularly stable cell growth is ensured independent on the type and size of groups A and B, the linking group and the cell adhesion factor.

The first comonomer is an oligo(alkylene glycol) substituted isocyanopeptide grafted with a linking group. Preferred examples of the linking group include azide (e.g oxanorbornadiene-based-azide), alkyne (e.g. dibenzocyclooctyne), thiol, vinylsulphone, maleimide, methyl methacrylate, ether, biotin, strepavidin, NH₂, COOH, OH, NHS-ester. Particularly preferred is azide.

An example of the first comonomer is shown in Formula (I), in which the linking group is an azide.

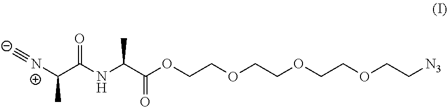 [SMILES: [C-]#[N+][C@H](C)C(=O)N[C@@H](C)C(=O)OCCOCCOCCOCCN=[N+]=[N-]]

The second comonomer is an oligo(alkylene glycol) substituted isocyanopeptide which is not grafted with a linking group or other groups, i.e. the side chain of the isocyanopeptide consists of an oligo(alkylene glycol). An example of the second comonomer is shown in Formula (II).

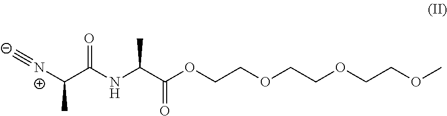

The first comonomer and the second comonomer are copolymerized in step (i). An oligo(alkylene glycol) substituted co-polyisocyanopeptide is obtained comprising linking groups along the polymer in the ratio of the first comonomer and the second comonomer.

A cell adhesion factor is attached to the copolymer via a spacer unit. First, a reactant of a spacer unit and a cell adhesion factor is made. An example of the spacer unit is shown in Formula (III).

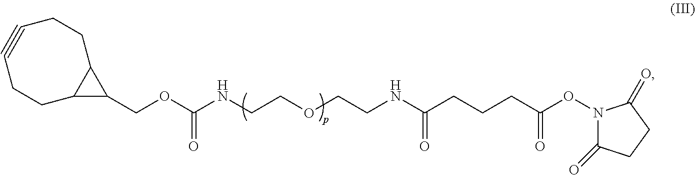

where p is 1 to 10.

In this example, group A is

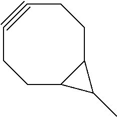

group B is

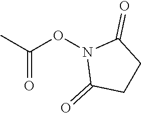

group L is

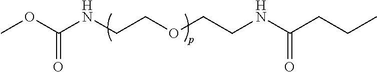

An example of the cell adhesion factor is shown in Formula (IV), which is a pentapeptide composed of glycine, L-arginine, glycine, L-aspartic acid, and serine (GRGDS).

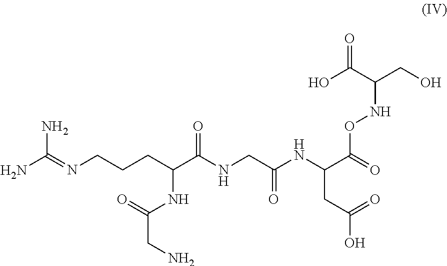 [SMILES: NCC(=O)NC(CCCN=C(N)N)C(=O)NCC(=O)NC(CC(=O)O)C(=O)ONC(CO)C(=O)O]

The reactant of the spacer unit of (III) and the cell adhesion factor of (IV) is shown in Formula (V).

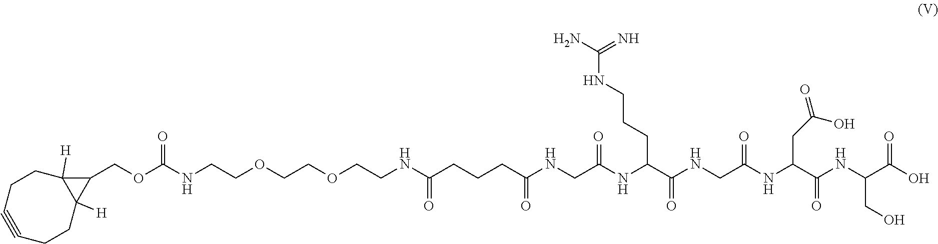 [SMILES: N=C(N)NCCCC(NC(=O)CNC(=O)CCCC(=O)NCCOCCOCCNC(=O)OCC1C2CCC#CCCC21)C(=O)NCC(=O)NC(CC(=O)O)C(=O)NC(CO)C(=O)O]

In step ii) of the invention, the reactant (e.g. formula (V)) of a spacer unit and a cell adhesion factor is reacted with the copolymer obtained by step i). The linking group reacts with the part of the reactant corresponding to the spacer unit. Accordingly, the final co-polyisocyanopeptide comprises cell adhesion units along the polymer in the ratio of the first comonomer and the second comonomer. An example of the final co-polyisocyanopeptide is represented by Formula (VI):

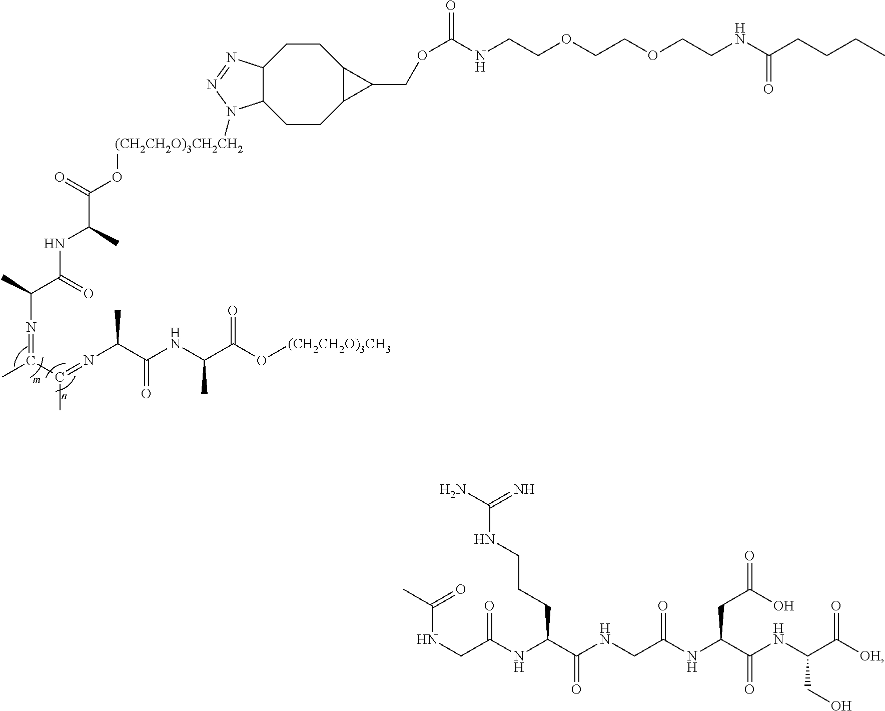

where m:n is the ratio of the first comonomer to the second comonomer.

Preferably, the cell adhesion factor is chosen from the group consisting of a sequence of amino acids such as RGD, GRGDS , rhrVEGF-164 and rhrbFGF.

The cell adhesion unit is positioned at a distance from the isocyanopeptide polymer backbone by the use of the spacer unit.

These embodiments of the present invention provide a cell culture of a hydrogel having a selective stiffness as well as controlled spacial distribution and density of cell adhesion points. The co-polymerisation results in a statistical distribution of the cell adhesion group along the copolymer in the ratio of the first comonomer and the second comonomer. The ratio between the first comonomer and the second comonomer can be tuned to control the distance between the cell adhesion factors along the polymer backbone of polyisocyanopeptide. The average distance between the cell adhesion factors along the polymer backbone is preferably at most 100 nm, preferably at most 70 nm. The average distance between the cell adhesion factors along the polymer backbone may e.g. be 1.1-60 nm. This range of the distance between the cell adhesion factors is suitable for anchoring the cells to be cultured to the cell culture. More preferably, the average distance between the cell adhesion factors is 8-30 nm.

The cell culture according to the invention is extremely advantageous in that the collection of the cultured cells is easy. The hydrogel used in the cell culture has a thermo-responsive character, i.e. it turns to liquid (the polymer solution) by cooling it to a temperature below the gelling temperature. Hence the collection of the cultured cells can be performed by only cooling the cell culture. After the hydrogel turns to liquid, the cells can be collected from the liquid without damaging the cultured cells.

It was determined that the cell adhesion factor cannot be directly attached to the oligo-alkylene glycol substituted isocyanopeptides to retain sufficient binding. This was solved by the use of a spacer according to the present invention. The spacer unit used according to the invention separates the cell adhesion factor from the polymer backbone of isocyanopeptides to eliminate steric blocking. The spacer decouples the motions of the cell adhesion factor from the polymer backbone and decoupling the motions allows the cell adhesion factor to dock efficiently into the integrin binding pocket. The spacer should be polar, water soluble, biocompatible and non-binding to the active site of the integrin, but can aid in auxiliary binding. The first monomer may be made by first preparing a second monomer and grafting it with a linking group. Alternatively, the first monomer and the second monomer may be made through different routes.

The molar ratio between the first comonomer and the second comonomer is between 1:500 and 1:30. Preferably, the molar ratio between the first comonomer and the second comonomer is between 1:400-1:35, 1:300-1:40 or 1:200-1:45. This range of the ratio between the first comonomer and the second comonomer gives an average distance of 8-30 nm between the cell adhesion units along the polymer backbone.

The gelation temperature of the the oligo(alkylene glycol) substituted co-polyisocyanopeptide is more than 18° C. and at most 38° C. such that the polymer solution is a liquid during steps a) and b) and is a hydrogel during step c). The gelation temperature is independent of the polymer concentration in the hydrogel. Rather it is dependent on the number of oligoalkylene glycol units in the side chain of the polymer.

Further details of the present invention are given below.

Comonomers

Functionalizing Isocyanopeptide with Oligo(Alkylene Glycol) Units.

The monomers are preferably based on a di-, tri-, tetra- or more peptidic motif substituted at the C terminal with the desired oligo(alkylene glycol) chains. The chains may be based on linear, branched or dendronized oligo(alkylene oxide). Preferably the chain is linear and is composed of ethylene glycol.

The peptidic segment can be of different compositions determined by the sequence of natural or non natural and expanded amino- acids or mixture thereof.

The monomers are derived from adequate oligo(alkylene glycol) fragments. A multi-steps peptidic coupling strategy is used to introduce successively the desired amino-acids. Following the introduction of the desired peptidic sequence, the N-terminus of the peptidic segment is formylated with an adequate formylation method. This formylation may include the treatment of the product with formyl salts, formic acid, or other formylating agents.

Some examples of formylation strategies make use of formate salts (such as sodium or potassium formate), alkyl formates (such as methyl-, ethyl-, or propyl-formate), formic acid, chloral and derivatives. The isocyanide is then formed by treating the formamide with an appropriate dehydration agent. An example of dehydratation strategy uses diphogene. Several examples of dehydratation agents that may also be used are phosgene and derivatives (di-, triphosgene,), carbodiimides, tosyl chloride, phosphorous oxachloride, triphenylphosphine/tetrachlorocarbon, [M. B. Smith and J. March “March's advanced organic chemistry” 5th edition, Wiley & Son eds., 2001, New York, USA, pp 1350-1351 and ref. herein;]

### Side Chains (Alkylene Glycol)

Examples of suitable alkylene glycols are ethylene-, propylene-, butylene- or pentylene glycol. Preferably the alkylene glycol is ethylene glycol.

Advantageous oligoethyleneglycol units are depicted below. In general, the term oligo refers to a number <10.

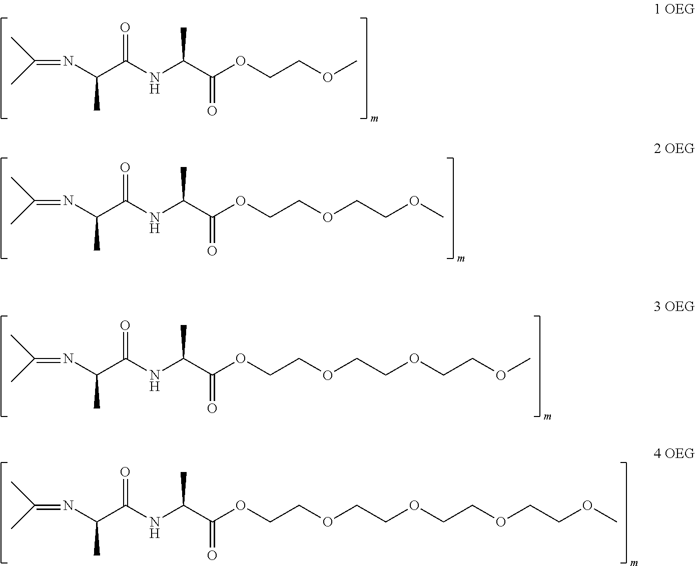

Preferably the isocyanopeptides are substituted with at least 3 ethylene glycol units to lead to water soluble materials after polymerization.

The second comonomer of the present invention is an oligo(alkylene glycol) isocyanopeptide as described above, without further grafting.

The first comonomer may consist of an isocyanopeptide having the same number of alkylene glycol units or may be a mixture of isocyanopeptides having different number of alkylene glycol units. Similarly, the second comonomer may consist of an isocyanopeptide having the same number of alkylene glycol units or may be a mixture of isocyanopeptides having different number of alkylene glycol units.

The first comonomer and the second comonomer are oligo(alkylene glycol) substituted isocyanopeptide, i.e. the number of the alkylene glycol units on the isocyanopeptide is 1 to 10. Preferably, the average of the number of the alkylene glycol units on the first comonomer and the second comonomer is at least 3 and at most 4.

The average of the alkylene glycol units on the first comonomer and the second comonomer is typically tuned by using a mixture of isocyanopeptides having different numbers of alkylene glycol units as the second comonomer. In preferred embodiments, the first comonomer is an isocyanopeptide having three alkylene glycol units and the second comonomer is a mixture of an isocyanopeptide having three alkylene glycol units and an isocyanopeptide having four alkylene glycol units.

The average of the number of the alkylene glycol units on the first comonomer and the second comonomer may be 3. The gelation temperature of 15-25° C. is typically obtained. The average of the number of the alkylene glycol units on the first comonomer and the second comonomer may be more than 3 and at most 3.5. The gelation temperature of 18-35° C. is typically obtained. The average of the number of the alkylene glycol units on the first comonomer and the second comonomer may be more than 3.5 and at most 5. The gelation temperature of 25-50° C. is typically obtained.

Preferably, the oligo(alkylene glycol) substituted co-polyisocyanopeptide has an elastic modulus of 10-5000 Pa, preferably 100-1000 Pa at a temperature of 35° C. as determined by rheology measurements. When the average of the number of the alkylene glycol units on the first comonomer and the second comonomer is at least 3 and at most 5, the hydrogel has such stiffness.

Polymerization

The oligo(alkylene glycol) isocyanopeptide monomer grafted with the linking group (first comonomer) and the oligo(alkylene glycol) isocyanopeptide monomers not grafted with the linking group (second comonomer) are mixed and subsequently copolymerized.

The copolymerization is preferably performed in the presence of an apolar solvent. Suitable apolar solvents may be selected from the group consisting of saturated hydrocarbon solvents and aromatic hydrocarbon solvents or mixtures thereof. Examples of apolar solvents are pentane, hexane, heptane, 2-methylbutane, 2-methylhexane, cyclohexane, and toluene, benzene xylenes or mixtures thereof. Preferably toluene is used in the polymerization. Preferably toluene is chosen for the polymerization process of oligo(ethylene glycol) isocyanopeptides where the oligo(ethylene glycol) part contains at least three ethylene glycol units.
Preferably the polymerization is carried out in the presence of a catalyst. The catalyst is preferably a nickel(II) salt. Example of Ni(II) salts are nickel(II) halides (e.g. nickel(II) chloride), nickel(II) perchlorate or tetrakis-(tertbutylisocyanide)nickel(II) perchlorate.

Other complexes and nickel salts might be used provided that they are soluble in the polymerization medium or initially dissolved in an adequate solvent which is miscible in the polymerization medium. General references describing some catalytic systems that may be used to polymerize the oligo(alkylene glycol)isocyanopeptides amy be found in Suginome M.; Ito Y; Adv Polym SC1 2004, 171 , 77-136; Nolte R. J. M.; Chem. Soc. Rev. 1994, 23(1), 11-19)]

Preferably the monomer concentration is chosen above 30 mmol/L and the catalyst/monomer ratio chosen between 1/100 and 1/10 000. Lowering the amount of nickel(II) (catalyst/monomer ratio below 1/1000) permits the preparation of materials exhibiting a substantial degree of polymerization [mean DP>500], which is desired for subsequent application of the polymers as macro-hydrogelators.

In a representative example, a millimolar solution of monomer in a nonpolar organic solvent or mixture of solvents is added to a nickel (II) catalyst dissolved in a polar solvent in a molar ratio of 1:50 up to 1:100,000 catalyst to monomer. In a sealed environment the mixture is vigorously stirred for 2 to 24 hrs. Once completed, the reaction mixture is evaporated and the crude product is dissolved in organic solvents and precipitated in diethylether or similar non-compatible organic solvents, giving the desired product.

Grafting of Reactant of Spacer Unit and Cell Adhesion Factor to Linking Group

Spacer Unit

The terminal groups A and B are preferably chosen such that the synthesis of the subsequent compound is possible without the need for deprotection or activation steps.

Preferred examples of group A of the spacer unit include azide (e.g oxanorbornadiene-based-azid), alkyne (e.g. dibenzocyclooctyne), thiol, vinylsulphone, maleimide, methyl methacrylate, ether, biotin, strepavidin, NH₂, COOH, OH, NHS-ester. Particularly preferred is alkyne.

Preferred examples of group B of the spacer unit include azide (e.g oxanorbornadiene-based-azid), alkyne (e.g. dibenzocyclooctyne), thiol, vinylsulphone, maleimide, methyl methacrylate, ether, biotin, strepavidin, NH₂, COOH, OH, NHS-ester. Particularly preferred is NHS-ester or malemide.

Preferably, the group A of the spacer unit is represented by formula (VII):

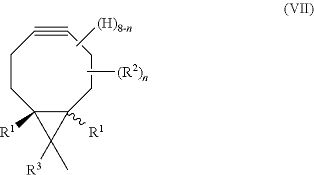

wherein: n is 0 to 8;

R³ is selected from the group consisting of [(L)ₚ-Q], hydrogen, halogen, C₁-C₂₄ alkyl groups, C₆- C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted with one or more substituents independently selected from the group consisting of C₁-C₁₂ alkyl groups, C₂-C₁₂ alkenyl groups, C₂-C₁₂ alkynyl groups, C₃-C₁₂ cycloalkyl groups, C₁-C₁₂ alkoxy groups, C₂-C₁₂ alkenyloxy groups, C₂-C₁₂ alkynyloxy groups, C₃-C₁₂ cycloalkyloxy groups, halogens, amino groups, oxo groups and silyl groups, wherein the alkyl groups, alkenyl groups, alkynyl groups, cycloalkyl groups, alkoxy groups, alkenyloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S, wherein the silyl groups are represented by the formula (R⁴)₃Si—, wherein R⁴ is independently selected from the group consisting of C₁-C₁₂ alkyl groups, C₂-C₁₂ alkenyl groups, C₂-C₁₂ alkynyl groups, C₃-C₁₂ cycloalkyl groups, C₁-C₁₂ alkoxy groups, C₂-C₁₂ alkenyloxy groups, C₂-C₁₂ alkynyloxy groups and C₃-C₁₂ cycloalkyloxy groups, wherein the alkyl groups, alkenyl groups, alkynyl groups, cycloalkyl groups, alkoxy groups, alkenyloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S;

R¹ is independently selected from the group consisting of hydrogen, C₁-C₂₄ alkyl groups, C₆-C₂₄ (hetero)aryl groups, C₇-C₂₄ alkyl(hetero)aryl groups and C₇-C₂₄ (hetero)arylalkyl groups; and

R² is independently selected from the group consisting of halogen, —OR⁶, —NO₂, —CN, —S(O)₂R⁶, C₁-C₁₂ alkyl groups, C₁-C₁₂ aryl groups, C₁-C₁₂ alkylaryl groups and C₁-C₁₂ arylalkyl groups, wherein R⁶ is as defined above, and wherein the alkyl groups, aryl groups, alkylaryl groups and arylalkyl groups are optionally substituted.

Preferably, n=0.

Preferably, R1 is hydrogen.

Preferably, R3 is hydrogen.

Preferably, the group B of the spacer unit is represented by formula (VIII):

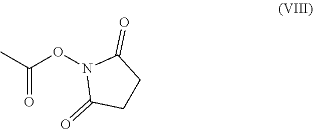

Preferably, the spacer unit comprises the group A of formula (VII) and the group B of formula (VIII).

Examples of the suitable spacer unit include the compounds represented by formula (IX):

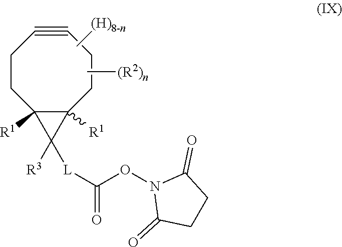

wherein R1, R2, R3 and n are as defined above and
L is preferably selected from the group represented by formula (X-1), (X-2, (X-3):

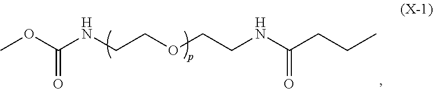

where p is 1 to 10, preferably 2 to 5,

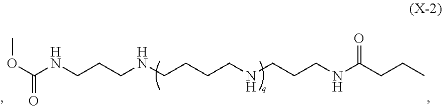

where q is 1 to 9 preferably 2 to 5,

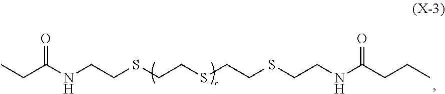

where r is 1 to 10, preferably 2 to 5.

Preferably, the spacer unit is represented by Formula (XI).

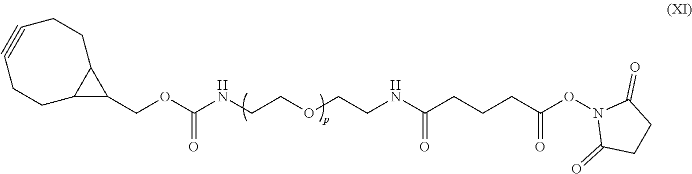

wherein p is 1 to 10, preferably 2 to 5, more preferably 2.

Other examples of the suitable spacer unit include fused cyclooctyne compounds described in WO2011/136645, which is incorporated herein by reference. Accordingly, a possible spacer unit is selected from the compound of the Formula (IIa, (IIb) or (IIc):

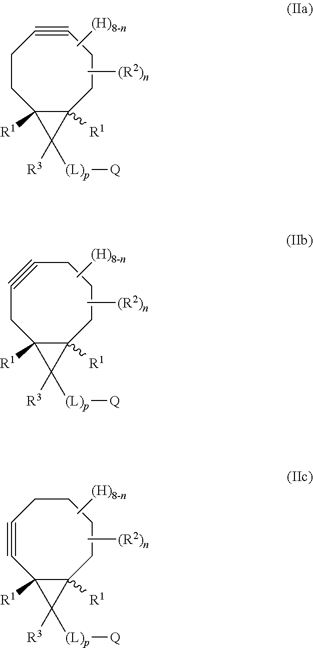

wherein:

n is 0 to 8;

p is 0 or 1;

R³ is selected from the group consisting of [(L)ₚ-Q], hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted with one or more substituents independently selected from the group consisting of C₁-C₁₂ alkyl groups, C₂-C₁₂ alkenyl groups, C2-C₁₂ alkynyl groups, C₃-C₁₂ cycloalkyl groups, C₁-C₁₂ alkoxy groups, C₂-C₁₂ alkenyloxy groups, C₂-C₁₂ alkynyloxy groups, C₃-C₁₂ cycloalkyloxy groups, halogens, amino groups, oxo groups and silyl groups, wherein the alkyl groups, alkenyl groups, alkynyl groups, cycloalkyl groups, alkoxy groups, alkenyloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S, wherein the silyl groups are represented by the formula (R⁴)₃Si—, wherein R⁴ is independently selected from the group consisting of C₁-C₁₂ alkyl groups, C₂-C₁₂ alkenyl groups, C₂-C₁₂ alkynyl groups, C₃-C₁₂ cycloalkyl groups, C₁-C₁₂ alkoxy groups, C₂-C₁₂ alkenyloxy groups, C₂-C₁₂ alkynyloxy groups and C₃-C₁₂ cycloalkyloxy groups, wherein the alkyl groups, alkenyl groups, alkynyl groups, cycloalkyl groups, alkoxy groups, alkenyloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S;

L is a linking group selected from linear or branched C₁-C₂₄ alkylene groups, C₂-C₂₄ alkenylene groups, C₂-C₂₄ alkynylene groups, C₃-C₂₄ cycloalkylene groups, C₅-C₂₄ cycloalkenylene groups, C₈-C₂₄ cycloalkynylene groups, C₇-C₂₄ alkyl(hetero)arylene groups, C₇-C₂₄ (hetero)arylalkylene groups, C₈-C₂₄ (hetero)arylalkenylene groups, C₉-C₂₄ (hetero)arylalkynylene groups, the alkylene groups, alkenylene groups, alkynylene groups, cycloalkylene groups, cycloalkenylene groups, cycloalkynylene groups, alkyl(hetero)arylene groups, (hetero)arylalkylene groups, (hetero)arylalkenylene groups and (hetero)arylalkynylene groups optionally being substituted with one or more substituents independently selected from the group consisting of C₁-C₁₂ alkyl groups, C₂-C₁₂ alkenyl groups, C₂-C₁₂ alkynyl groups, C₃-C₁₂ cycloalkyl groups, C₅-C₁₂ cycloalkenyl groups, C₈-C₁₂ cycloalkynyl groups, C₁-C₁₂ alkoxy groups, C₂-C₁₂ alkenyloxy groups, C₂-C₁₂ alkynyloxy groups, C₃-C₁₂ cycloalkyloxy groups, halogens, amino groups, oxo and silyl groups, wherein the silyl groups can be represented by the formula (R⁴)₃Si—, wherein R⁴ is defined as above;

Q is a functional group selected from the group consisting of hydrogen, halogen, R⁶, —CH═C(R⁶)₂, —C≡CR⁶, —[C(R⁶)₂C(R⁶)₂O]_{q}—R⁶, wherein q is in the range of 1 to 200, —CN, —N₃, —NCX, —XCN, —XR⁶, —N(R⁶)₂, -−+N(R⁶)₃, —C(X)N(R⁶)₂, —C(R⁶)₂XR⁶, —C(X)R⁶, —C(X)XR⁶, —S(O)R⁶, —S(O)2R⁶, —S(O)OR⁶, —S(O)2OR⁶, —S(O)N(R⁶)₂, —S(O)₂N(R⁶)₂, —OS(O)R⁶, —OS(O)₂R⁶, —OS(O)OR⁶, —OS(O)₂OR⁶, —P(O)(R⁶)(OR⁶), —P(O)(OR⁶)₂, —OP(O)(OR⁶)₂, —Si(R⁶)₃, —XC(X)R⁶, —XC(X)XR⁶, —XC(X)N(R⁶)₂, —N(R⁶)C(X)R⁶, —N(R⁶)C(X)XR⁶ and —N(R⁶)C(X)N(R⁶)₂, wherein X is oxygen or sulphur and wherein R⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;

R¹ is independently selected from the group consisting of hydrogen, C₁-C₂₄ alkyl groups, C₆-C₂₄ (hetero)aryl groups, C₇-C₂₄ alkyl(hetero)aryl groups and C₇-C₂₄ (hetero)arylalkyl groups; and

R² is independently selected from the group consisting of halogen, —OR⁶, —NO₂, —CN, —S(O)₂R⁶, C₁-C₁₂ alkyl groups, C₁-C₁₂ aryl groups, C₁-C₁₂ alkylaryl groups and C₁-C₁₂ arylalkyl groups, wherein R⁶ is as defined above, and wherein the alkyl groups, aryl groups, alkylaryl groups and arylalkyl groups are optionally substituted.

Cell Adhesion Factor

The cell adhesion factor supports the binding of cells to the gel. The cell adhesion factor preferably is a sequence of amino acids. Examples of amino acids that advantageously may be used in the present invention are N-protected Alanine, Arginine, Asparagines, Aspartic acid, Cysteine, Glutamic acid, Glutamine, Glycine, Histidine, Isoleucine, Leucine, Lysine, Methionine, Phenylalanine, Proline, Serine, Threonine, Thryptophan, Tyrosine, Valine. Suitable sequences of amino acids include peptides such as RGD, GRGDS, IKVAV, KQAGDV and GRGDSP. The cell adhesion factor may also be a growth factor such as VGEF and BFGF. The cell adhesion factor may also be glycoproteins or mucins.

The spacer unit and the cell adhesion factor are reacted. The reactant may be grafted to the linking group of the copolymer by copper free SPAAC reaction.

A hydrogel is made from the copolymer as obtained by gelling with a suitable cell culture medium. The hydrogel is a three dimensional hydrogel.

Stem Cells

Preferred stem cells are stem cells chosen from the group consisting of human adipose stem cells and human mesenchymal stem cells, e.g. bone marrow derived mesenchymal stem cells, adipose derived mesenchymal stem cells, umbellical cord derived mesenchymal stem cells, amniotic fluid mesenchymal stem cells, embryonic stem cells and induced pluripotent stem cells.

Cell Culture

The cell culture according to the invention comprises the hydrogel as described above.

The cell culture is a three dimensional porous scaffold.

Guidelines for choosing a cell culture medium and cell culture conditions are well known and are for instance provided in Chapter 8 and 9 of Freshney, R. I. Culture of animal cells (a manual of basic techniques), 4th edition 2000, Wiley-Liss and in Doyle, A. , Griffiths, J. B., Newell, D. G. Cell & Tissue culture: Laboratory Procedures 1993, John Wiley & Sons.

Generally, a cell culture medium for (mammalian) cells comprises salts, amino acids, vitamins, lipids, detergents, buffers, growth factors, hormones, cytokines, trace elements, carbohydrates and other organic nutrients, dissolved in a buffered physiological saline solution. Examples of salts include magnesium salts, for example MgCl₂.6H₂O, MgSO₄ and MgSO₄.7H₂O iron salts, for example FeSO₄.7H₂O, potassium salts, for example KH₂PO₄, KCl; sodium salts, for example NaH₂PO₄, Na₂HPO₄ and calcium salts, for example CaCl₂.2H₂O. Examples of amino acids are all 20 known proteinogenic amino acids, for example hystidine, glutamine, threonine, serine, methionine. Examples of vitamins include: ascorbate, biotin, choline.Cl, myo-inositol, D-panthothenate, riboflavin. Examples of lipids include: fatty acids, for example linoleic acid and oleic acid; soy peptone and ethanol amine. Examples of detergents include Tween 80 and Pluronic F68. An example of a buffer is HEPES. Examples of growth factors/hormones/cytokines include IGF, hydrocortisone and (recombinant) insulin. Examples of trace elements are known to the person skilled in the art and include Zn, Mg and Se. Examples of carbohydrates include glucose, fructose, galactose, sucrose and pyruvate.

It is preferred that the cell culturing medium contains a serum. For example a serum which is selected from fetal bovine serum (FBS), fetal calf serum (FCS), horse serum or human serum. The serum may be present between 1 and 15 wt %, relative to the amount of cell culturing media, or between 3 and 12 wt %.

The culture medium may be supplemented with growth factors, metabolites, etc. Depending on the preferred differentiation, different media can be used.

Cell culturing media may comprise e.g. MEM alpha modification, Dulbecco's MEM, Iscove's MEM, 199 medium, CMRL 1066, RPMI 1640, F12, F10, DMEM, Waymouth's MB752/1, VEGM, OST and McCoy's 5A.

Preferably the cell culturing medium comprises αMEM, DMEM, VEGM and/or OST.

The cell culturing medium for osteogenic differentiation may comprise β-glycerosphosphate, L-ascorbic acid and dexamethasone.The cell culturing medium for osteogenic differentiation may be a minimum essential medium supplemented with β-glycerosphosphate, L-ascorbic acid and dexamethasone. The minimum essential medium may e.g. be αMEM medium, which is αMEM medium (=minimum essential medium eagle-α modification, Gibco, USA) supplemented with 10% (v/v) of fetal calf serum and 1% (v/v) penicilin/streptomycin (100U/100 μg/mL, Gibco, USA). Other types of minimum essential medium are known as DMEM and RPMI.

The cell culturing medium for osteogenic differentiation may be αMEM supplemented with 10 mM β-glycerosphosphate (Sigma, Germany, Cat No G9422), 50 μg/mL of L-ascorbic acid (Sigma, Germany, Cat No A8960) and 10⁻⁸ M dexamethasone (Sigma, Germany, Cat. No D4902).

Cell culturing media for inducing vascularization or adipogenic differentiation may comprise e.g. MEM alpha modification, Dulbecco's MEM, Iscove's MEM, 199 medium, CMRL 1066, RPMI 1640, F12, F10, DMEM, Waymouth's MB752/1, VEGM and McCoy's 5A.

Preferably the cell culturing medium for inducing vascularization or adipogenic differentiation comprises αMEM, DMEM and/or VEGM.

The optimal conditions under which the cells are cultured can easily be determined by the skilled person. For example, the pH, temperature, dissolved oxygen concentration and osmolarity of the cell culture medium are in principle not critical and depend on the type of cell chosen. Preferably, the pH, temperature, dissolved oxygen concentration and osmolarity are chosen such that these conditions optimal for the growth and productivity of the cells. The person skilled in the art knows how to find the optimal pH, temperature, dissolved oxygen concentration and osmolarity. Usually, the optimal pH is between 6.6 and 7.6, the optimal temperature between 30 and 39° C., for example a temperature from 36 to 38° C., preferably a temperature of about 37° C.; the optimal osmolarity between 260 and 400 mOsm/kg.

Osteogenic Differentiation

According to one embodiment, the invention provides a method for inducing osteogenic differentiation of stem cells, comprising the steps of:

a) Mixing a cell culturing medium for osteogenic differentiation with an oligo(alkylene glycol) substituted co-polyisocyanopeptide at a temperature between 0 and 18° C. to obtain a polymer solution;

b) Mixing the polymer solution with stem cells at a temperature between 0 and 18° C. to obtain a cell culture solution;

c) Allowing the cell culture solution to warm to a temperature between 30 and 38° C. to form a cell culture comprising a hydrogel and allow the stem cells to differentiate,

wherein the concentration of the polyisocyanopeptide in the polymer solution is 1-5 mg/ml,

wherein the average length of the polyisocyanopeptide is 50-750 nm as determined by AFM,

wherein the cell density of the stem cells in the cell culture solution is 0.3*10⁶-1*10⁶ cells/Ml,

wherein the hydrogel has a critical stress σ_{c} of 13-30 Pa, wherein the critical stress σ_{c} is a stress which marks an onset of a strain stiffening,

wherein the hydrogel has a storage modulus G′ measured at 37° C. of 50-1000 Pa, preferably between 70-450 Pa, more preferably between 72-400 Pa,

wherein the polyisocyanopeptide has a cell adhesion factor covalently bound to the polyisocyanopeptide and/or wherein the cell culturing medium comprises fibrin,

wherein when the polyisocyanopeptide has a cell adhesion factor covalently bound to the polyisocyanopeptide, the average distance between the cell adhesion factors along the polyisocyanopeptide backbone is 10-50 nm.

Preferably, the viscosity average molecular weight (Mv) of the polyisocyanopeptide is 100-1000 kg/mol. More preferably, the viscosity average molecular weight (Mv) of the polyisocyanopeptide is 500-1000 kg/mol for osteogenic differentiation.

Preferably, the average length of the polyisocyanopeptide is 200-700 nm, more preferably 250-680 nm, more preferably 280-650 nm, as determined by AFM.

The relationship between the viscosity average molecular weight (Mv) and the average length of the polyisocyanopeptide can be derived from table 1. The molecular weight of 300 kg/mol corresponds to the length of about 180 nm. The molecular weight of 685 kg/mol corresponds to the length of about 434 nm.

It was found that an increase in the molecular weight of the polyisocyanopeptide results in a substantially linear increase in the critical stress while maintaining the storage modulus G′ within a relatively narrow range. Accordingly, it is possible according to the invention to induce osteogenic differentiation of stem cells in a highly accurate manner while maintaining the storage modulus of the hydrogel within the optimal range.

Accordingly, in particularly preferred embodiments, the storage modulus G′ measured at 37° C. is 200-400 Pa and the viscosity average molecular weight (Mv) of the polyisocyanopeptide is between 100 and 1000 kg/mol, preferably 500-1000 kg/mol.

Vascularization

According to a second embodiment of the invention provides a method for inducing vascularization of stem cells, comprising the steps of:

a) Mixing a cell culturing medium for vascularization with an oligo(alkylene glycol) substituted co-polyisocyanopeptide at a temperature between 0 and 18° C. to obtain a polymer solution;

b) Mixing the polymer solution with stem cells at a temperature between 0 and 18° C. to obtain a cell culture solution;

c) Allowing the cell culture solution to warm to a temperature between 30 and 38° C. to form a cell culture comprising a hydrogel and allow the stem cells to differentiate,

wherein the concentration of the polyisocyanopeptide in the polymer solution is 1-5 mg/ml,

wherein the average length of the polyisocyanopeptide is 50-750 nm as determined by AFM,

wherein the cell density of the stem cells in the cell culture solution is 0.3*10⁶-1*10⁶ cells/ml,

wherein the hydrogel has a critical stress σ_{c} of 2-30 Pa, preferably 7-12 Pa, wherein the critical stress is a stress which marks an onset of a strain stiffening,

wherein the hydrogel has a storage modulus G′ measured at 37° C. of 50-1000 Pa, preferably between 70-400 Pa,

wherein the polyisocyanopeptide has a cell adhesion factor covalently bound to the polyisocyanopeptide and/or wherein the cell culturing medium comprises fibrin,

wherein when the polyisocyanopeptide has a cell adhesion factor covalently bound to the polyisocyanopeptide, the average distance between the cell adhesion factors along the polyisocyanopeptide backbone is 10-50 nm.

Preferably, the viscosity average molecular weight (Mv) of the polyisocyanopeptide is 100-1000 kg/mol. More preferably, the viscosity average molecular weight (Mv) of the polyisocyanopeptide is 200-700 kg/mol, or 300-600 kg/mol for vascularization.

The average length of the polyisocyanopeptide is generally 50-750 nm as determined by AFM. For vascularization, preferably, the average length of the polyisocyanopeptide is 50-400 nm, more preferably 70-300 nm, more preferably 80-250 nm, as determined by AFM.

The relationship between the viscosity average molecular weight (Mv) and the average length of the polyisocyanopeptide can be derived from Table 1. The molecular weight of about 300 kg/mol corresponds to the length of about 180 nm. The molecular weight of 685 kg/mol corresponds to the length of 434 nm.

It was found that an increase in the molecular weight of the polyisocyanopeptide results in a substantially linear increase in the critical stress while maintaining the storage modulus G′ within a relatively narrow range. Accordingly, it is possible according to the invention to induce vascularization of stem cells in a highly accurate manner while maintaining the storage modulus of the hydrogel within the optimal range.

Accordingly, in particularly preferred embodiments, the storage modulus G′ measured at 37° C. is 70-300 Pa and the viscosity average molecular weight (Mv) of the polyisocyanopeptide is between 100 and 1000 kg/mol, preferably 200-700 kg/mol or 300-600 kg/mol.

Adipogenic Differentiation

According to a third embodiment the invention provides a method for inducing adipogenic differentiation of stem cells, comprising the steps of:

a) Mixing a cell culturing medium for adipogenic differentiation with an oligo(alkylene glycol) substituted co-polyisocyanopeptide at a temperature between 0 and 18° C. to obtain a polymer solution;

b) Mixing the polymer solution with stem cells at a temperature between 0 and 18° C. to obtain a cell culture solution;

c) Allowing the cell culture solution to warm to a temperature between 30 and 38° C. to form a cell culture comprising a hydrogel and allow the stem cells to differentiate,

wherein the concentration of the polyisocyanopeptide in the polymer solution is 1-5 mg/ml,

wherein the average length of the polyisocyanopeptide is 50-750 nm as determined by AFM,

wherein the cell density of the stem cells in the cell culture solution is 0.3*10⁶-1*10⁶ cells/ml,

wherein the hydrogel has a critical stress σ_{c} of 2-30 Pa, preferably 7-23 Pa or 8-20 Pa, wherein the critical stress is a stress which marks an onset of a strain stiffening,

wherein the hydrogel has a storage modulus G′ measured at 37° C. of 50-1000 Pa, preferably between 70-450 Pa, more preferably between 72-400 Pa,

wherein the polyisocyanopeptide has a cell adhesion factor covalently bound to the polyisocyanopeptide and/or wherein the cell culturing medium comprises fibrin,

wherein when the polyisocyanopeptide has a cell adhesion factor covalently bound to the polyisocyanopeptide, the average distance between the cell adhesion factors along the polyisocyanopeptide backbone is 10-50 nm.

Preferably, the viscosity average molecular weight (Mv) of the polyisocyanopeptide is 100-1000 kg/mol. More preferably, the viscosity average molecular weight (Mv) of the polyisocyanopeptide is 200-700 kg/mol, or 300-600 kg/mol for adipogenic differentiation.

The average length of the polyisocyanopeptide is generally 50-750 nm as determined by AFM. For adipogenic differentiation, preferably, the average length of the polyisocyanopeptide is 50-400 nm, more preferably 70-300 nm, more preferably 80-250 nm, as determined by AFM.

The relationship between the viscosity average molecular weight (Mv) and the average length of the polyisocyanopeptide can be derived from Table 1. The molecular weight of about 300 kg/mol corresponds to the length of about 180 nm. The molecular weight of 685 kg/mol corresponds to the length of 434 nm.

It was found that an increase in the molecular weight of the polyisocyanopeptide results in a substantially linear increase in the critical stress while maintaining the storage modulus G′ within a relatively narrow range. Accordingly, it is possible according to the invention to induce adipogenic differentiation of stem cells in a highly accurate manner while maintaining the storage modulus of the hydrogel within the optimal range.

Accordingly, in particularly preferred embodiments, the storage modulus G′ measured at 37° C. is 70-450 Pa and the viscosity average molecular weight (Mv) of the polyisocyanopeptide is between 100 and 1000 kg/mol, preferably 200-700 kg/mol or 300-600 kg/mol.

The invention also relates to the use of the cell culture for in vitro differentiation of stem cells.

The invention further relates to the use of the cell culture as a medicament.

The invention further relates to the use of the cell culture for in vivo differentiation of stem cells.

Although the invention has been described in detail for purposes of illustration, it is understood that such detail is solely for that purpose and variations can be made therein by those skilled in the art without departing from the spirit and scope of the invention as defined in the claims.

It is further noted that the invention relates to all possible combinations of features described herein, preferred in particular are those combinations of features that are present in the claims.

It is further noted that the term ‘comprising’ does not exclude the presence of other elements. However, it is also to be understood that a description on a product comprising certain components also discloses a product consisting of these components. Similarly, it is also to be understood that a description on a process comprising certain steps also discloses a process consisting of these steps.

### BRIEF DESCRIPTION OF THE DRAWINGS

FIG. 1 shows the relationship between differential modulus K′ and the applied stress for various hydrogels.

FIG. 2a shows the reaction of a non-functionalized monomer 1 and an azide appended monomer 2.

FIG. 2b shows the formation of BCN-GRGDS conjugate.

FIG. 2c shows the formation of polymer-peptide conjugates.

FIG. 2d shows the relationship of the stiffness G′ (Pa) of the hydrogel according to the invention and the temperature (° C.). The onset of gelation temperature was observed to be ˜15° C.

FIG. 2e shows the storage modulus G₀ (Pa) of hydrogels P1-P6 in relation to the mean polymer length. The storage modulus (Gₒ) remains fairly constant (0.2-0.4 kPa) at 37° C. as a function of polymer length.

FIG. 2f shows the critical stressσ_{c} (Pa) in relation to the mean polymer length. The critical stress varies linearly as a function of polymer length.

FIG. 3a-c show images of the cells.

FIG. 3d-i show results of various tests for polymers P1-P6.

FIG. 4a-b shows the influence of the critical stress of the hydrogel to the stem cell differentiation.

FIG. 5 shows the critical stresses (94 _{c}) of P1-P6 α-MEM gels.

FIG. 6 shows the relationship between the molecular weight of the polyisocyanopeptides used according to the invention and the critical stress of the hydrogel made using the polyisocyanopeptides.

FIGS. 7-9 show the expression of osteogenic (RUNX2, ALP, FOSB and DLX5), endothelial (EDF1, VWF, KDR/FLK-1, and CD31), adipogenic (PPARγ, CEBPB, LPL and FABP4) specific genes for stem cells grown in osteogenic, adipogenic and endothelial media, respectively; P7=soft, P8=medium, P9=hard.

FIG. 10 shows the growth of stem cells in alfa MEM (reference experiment); P7=soft, P8=standard, P9=hard.

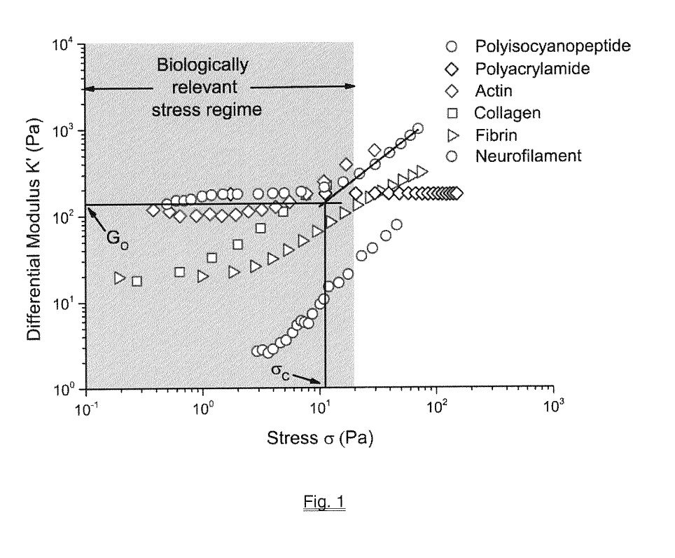

### DETAILED DESCRIPTION OF THE INVENTION

Experiment 1

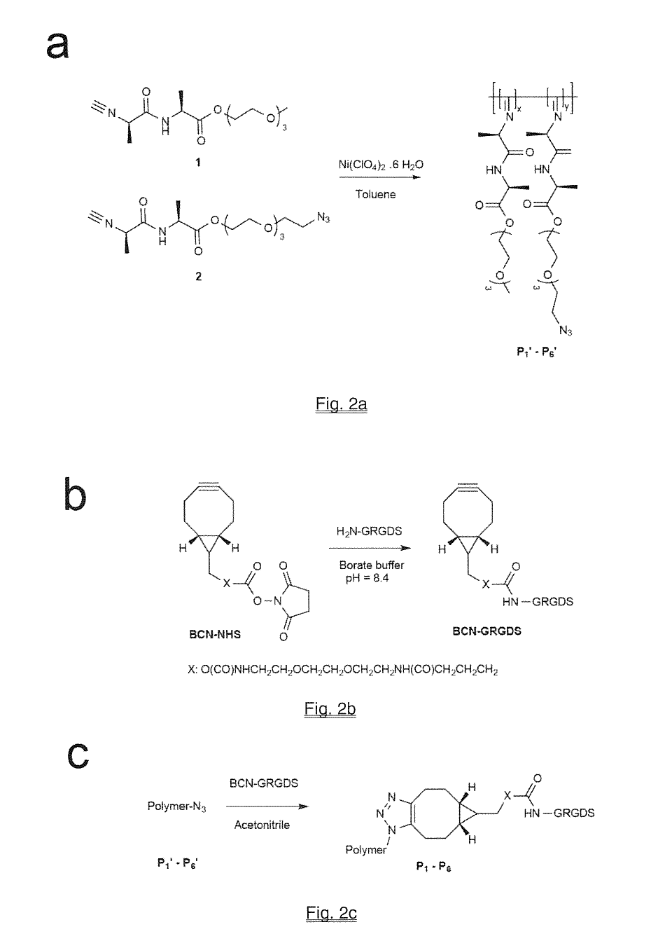

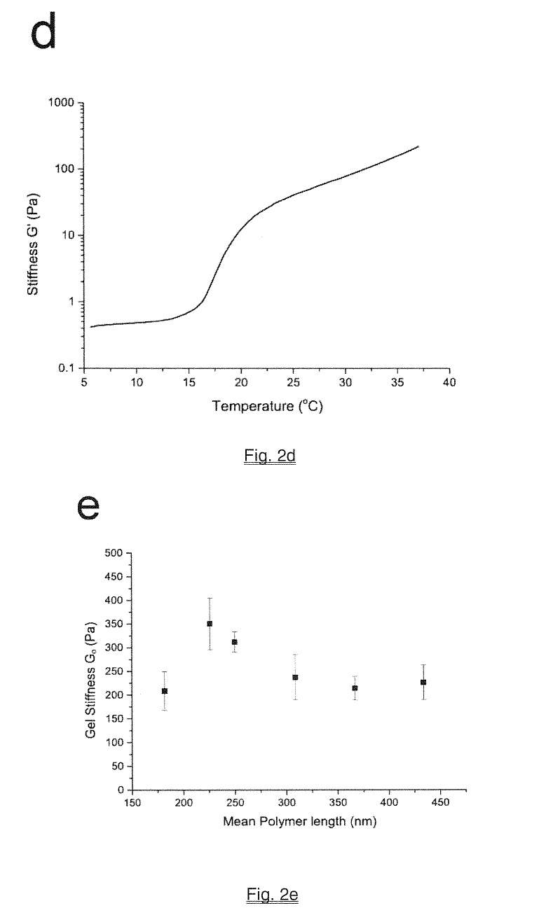

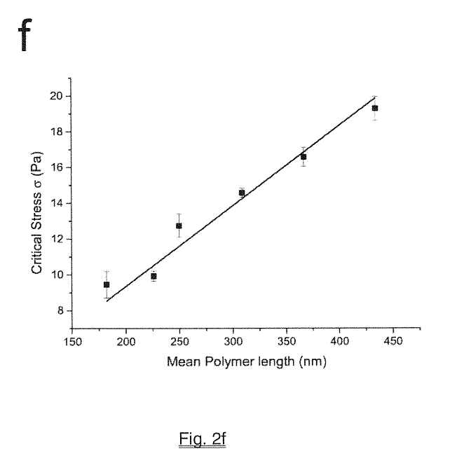

Polyisocyanopeptides (P1′-P6′) were synthesized by a nickel (II)-catalyzed co-polymerization of triethylene glycol functionalized isocyano-(D)-alanyl-(L)-alanine monomer 1 and the azide-appended monomer 2 (FIG. 2a), with the molar ratio of 1/2=100, resulting in polymers with one azide functionality every 14-18 nm of the polymer chain, as determined by reacting a strained rhodamine dye to the azides (Table 1 and Methods).

The catalyst to monomer molar ratio was varied from 1:1000 to 1:8000, to obtain polymers of increasing molecular weight (determined by viscosity measurements, Table 1) (P1′-P6′). These azide functionalized polymers were then subjected to strain-promoted click reaction with BCN-GRGDS (BCN: Bicyclo[6.1.0]non-4-yn-9-ylmethyl) to obtain cell adhesive GRGDS functionalized polymers P1-P6 (FIG. 2b-c and Methods) of increasing chain lengths as determined by AFM (Table 1). Solutions of these polymers in α-MEM (Minimum Essential Medium) at a fixed concentration (2 mg/mL) formed transparent gels upon warming, above ˜15° C. (temperature sweep rheology, FIG. 2d).

The mechanical properties of the GRGDS functionalized polymer gels were investigated by rheological analysis. Temperature sweep experiments (heating up to 37° C.) followed by time sweep at 37° C. revealed that all the gels P1-P6 were soft and exhibited similar stiffnesses (0.2-0.4 kPa at 37° C.) (FIG. 2e). Recently, we reported that hydrogels of non-functionalized polyisocyanopeptide polymers show a biomimetic stress stiffening behavior (Kouwer, P. H. J. et al. Responsive biomimetic networks from polyisocyanopeptide hydrogels. Nature 493, 651-655 (2013). Using the same pre-stress protocol (Broedersz, C. P. et al. Measurement of nonlinear rheology of cross-linked biopolymer gels. Soft Matter 6, 4120 (2010)), the critical stresses (σ_{c}) of P1-P6 α-MEM gels were measured (FIG. 5). The critical stress (σ_{c}) for non-linear rheology behavior of these gels was found to increase linearly as a function of the polymer chain length (FIG. 2f and Table 1), from ˜9 Pa in the P1 gel (average polymer length: 182 nm) to ˜19 Pa in the P6 gel (average polymer length: 434 nm). Although it appears that there is a 1.5-fold increase in the mean gel stiffness when the mean polymer length is increased from ˜180 nm to ˜240 nm (FIG. 2e), this difference is small in the context of cellular perception of bulk stiffness¹⁷. Regarding the critical stress values, the error range is smaller and there appears to be a linear relationship between this parameter and the mean polymer length (FIG. 2f) which is why we consider the increase to be significant.

Effect of Stress-Stiffening on hMSC Commitment and Differentiation.

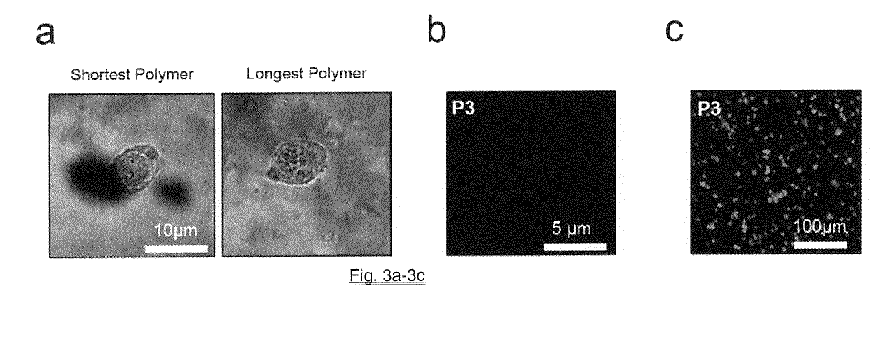

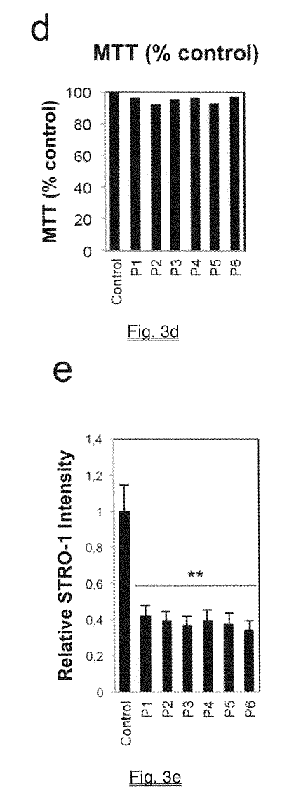

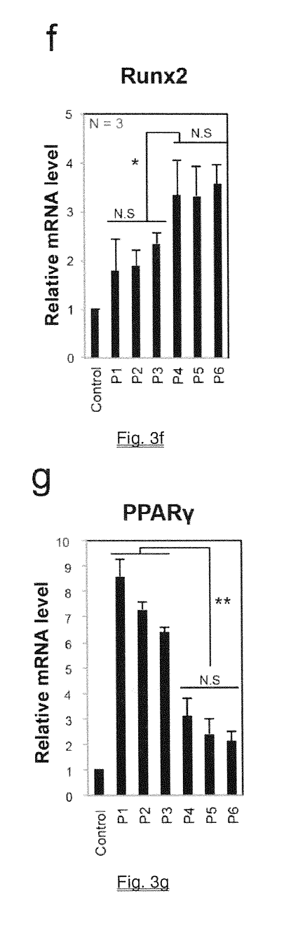

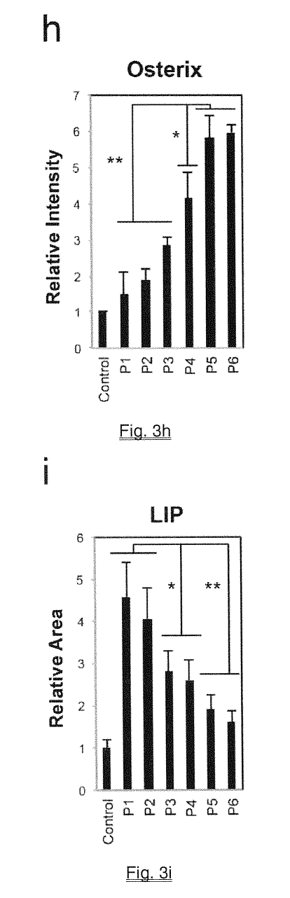

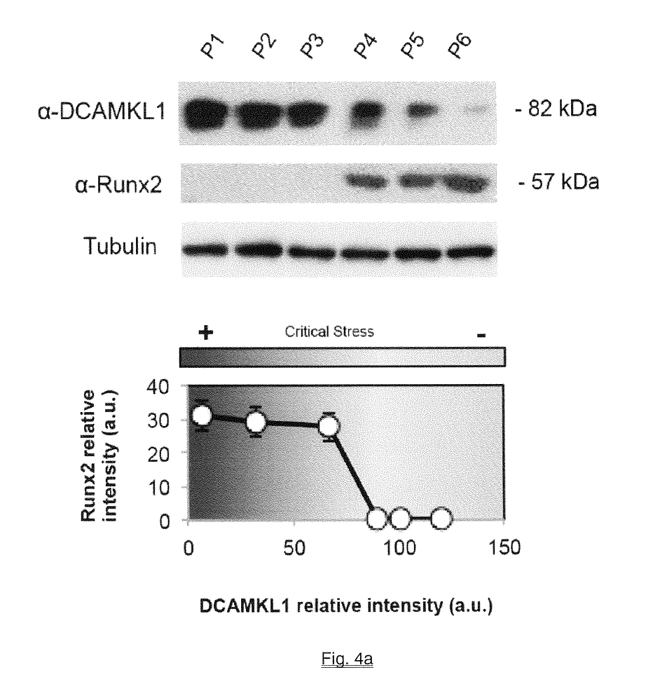

To investigate the effect of stress-stiffening on stem cell fate, hMSCs were mixed with a cold polymer solution (˜10° C.) in α-MEM, which was then warmed to 37° C. to form the 3D matrix with encapsulated hMSCs. The cells were homogeneously distributed throughout the gel as indicated by confocal microscopy. Investigation of hMSCs morphology after 36 h of culture for all of the gels (P1-P6) revealed that the cells remained spherical (FIG. 3a). These cells exhibited only limited cortical F-actin protrusions into the surrounding microenvironment (Phalloidin staining) and showed no significant modifications in their nuclear morphology as shown by a representative DAPI fluorescence image of the cell nucleus after 36 h of culture (FIG. 3b). Live/dead assay (calcein-AM and MTT) performed after 36 h of culture in growth media for all of the gels indicated excellent viability (>95%) of the encapsulated cells (FIG. 3c and d), as also confirmed by confocal microscopy. In addition, no significant cell proliferation could be detected for the various gels as determined by the PicoGreen assay. The lineage commitment of the gel encapsulated hMSCs after 96 h of culture in bipotential differentiation medium (1:1 v/v osteogenic and adipogenic media) was then investigated. Cells were first stained (immunofluorescence) for STRO-1, a mesenchymal stem cell specific marker. A significant decrease in the average STRO-1 expression was observed for the cells in all of the gels after 96 h of culture, indicating the onset of stem cell differentiation (FIG. 3e). The expression of osteogenic and adipogenic differentiation markers was then examined. For cells cultured in the gel with the lowest critical stress (σ_{c} ˜9.4 Pa, constructed from the shortest polymer P1) predominant adipogenic commitment was observed (Oil-red O staining, FIG. 3i). With increasing the critical stress (by increasing the polymer length), osteogenesis was progressively favored over adipogenesis, as demonstrated by immunofluorescent staining of Osterix, an osteogenic specific marker (FIG. 3h) and as determined from the mean percentages of osteogenic and adipogenic commitments in the various polymers (P1-P6). hMSCs cultured in the gel with the highest critical stress (P6) exhibited preferential osteogenic commitment. The predominant osteogenesis for the cells in the longer polymers (P4-P6) was further confirmed by differentiation tests after 3 weeks of culture.

Finally, the hMSCs osteogenic commitment was verified by analyzing the expression of the osteogenic biomarker Core-binding factor α1 (Cbfa-1), also called RUNX2 and the expression of the adipogenic biomarker PPARγ, by RT-PCR. We observed an increase in the RUNX2 gene expression with increasing the critical stress after 96 h of culture (FIG. 3f) in agreement with the immunofluorescence staining results. Increase in osteogenesis for the longer polymer gels has been further confirmed by the observed decrease in PPARγ gene expression as a function of the increasing critical stress after 96 h of culture (FIG. 3g).

To investigate the role of hMSCs-adhesive ligand interactions in the observed stem cell fate, we performed the cell commitment studies for RGD modified polymers P1, P3, P4 and P6 in the presence of antibodies recognizing specific integrin subunits (α1, 2, 3 and 5; β31 and 2) which block their interactions with the substrate bound RGD ligands. In the presence of these integrin blocking antibodies, osteogenic commitment was suppressed. However, adipogenic commitment was maintained for all the polymers. This result is in agreement with recent literature and highlights the importance of the interaction between integrin receptors and the RGD ligands for mediating the stress-stiffening induced commitment switch. Interestingly, the presence of blebbistatin (a small molecule inhibitor of actomyosin contractility showing high affinity and selectivity toward myosin II) inhibited the hMSCs commitment, with sternness maintenance observed for all the polymer gels, as revealed by the high levels of STRO-1 in the encapsulated cells. This suggests that the inhibition of actomyosin contraction interferes with the mechanisms of hMSCs commitment both towards adipogenesis and osteogenesis. This is most likely due to the fact that the cells could not apply any traction force for the microenvironmental mechanical (stress-stiffening) sensing. These results are consistent with previously published studies. Finally, in order to demonstrate the direct interaction between the hMCSs and the polymer-bound RGD in our system, the cell commitment studies for RGD modified polymers P1, P3, P4 and P6 were performed in the presence of soluble RGD ligands, which can block the interaction between the cells and the matrix by competing for the integrin binding sites. No significant osteogenic or adipogenic commitment could be detected indicating that integrin disengagement from the matrix bound RGD is interfering with the cell's ability to sense stress-stiffening. These data also imply that the cells in these gel culture systems need direct engagement with the bound RGD ligand, and not with the secreted ECM, for mediating the stress-stiffening induced commitment switch.

Although the macroscopic ligand density is kept constant in this study (one ligand every 14-18 nm of a polymer chain), the longer polymer chains (P4-P6) have almost 2-fold higher number of ligands per chain (20-26), as compared to the corresponding shorter chains (P1-P3: 13-18). This could indeed impact the extent of cell-mediated local ligand clustering. To study the effect of ligand-density on the observed hMSC commitment switch, the commitment study was performed as a function of ligand density (RGD every 7 nm, 28 nm and 70 nm) for gels of the shortest (P1) and the longest polymer (P6). Varying the ligand density for both of the polymers was found not to interfere with the cell differentiation outcome. These results suggest that stress-stiffening is the primary governing variable in our system, without excluding the possibility that cell-mediated ligand clustering is occurring. Our data demonstrate that hMSCs fate can be switched from adipogenesis to osteogenesis in a soft microenvironment (˜0.2-0.4 kPa), simply by increasing the critical stress for the onset of stress-stiffening.

Stress-Stiffening Mediated Stem Cell Differentiation Involves the Microtubule-Associated Protein DCAMKL1.

Several reports have implicated the cytoskeletal contractility and actin polymerization in the mechanotransduction pathway responsible for osteogenic differentiation on 2D substrates. In our study, a treatment with cytochalasin-D (inhibitor of actin polymerization) resulted in an overall decreased commitment of the cultured stem cells towards both osteogenesis and adipogenesis, suggesting a role of actin polymerization in the stress-stiffening mediated hMSCs differentiation in our system. Alternatively we also observed a decrease in hMSCs commitment after treatment with Taxol, a well-characterized microtubule-stabilizing agent, which is known to inhibit tubulin de-polymerization. Taxol treatment did not affect cell viability as indicated by a live/dead assay after 48 h and 96 h of culture. The effect of Taxol on the cell commitment outcome indicates that, in addition to actin, the microtubule dynamics could also be involved in the mechanotransduction pathways underlying hMSCs differentiation in our system.

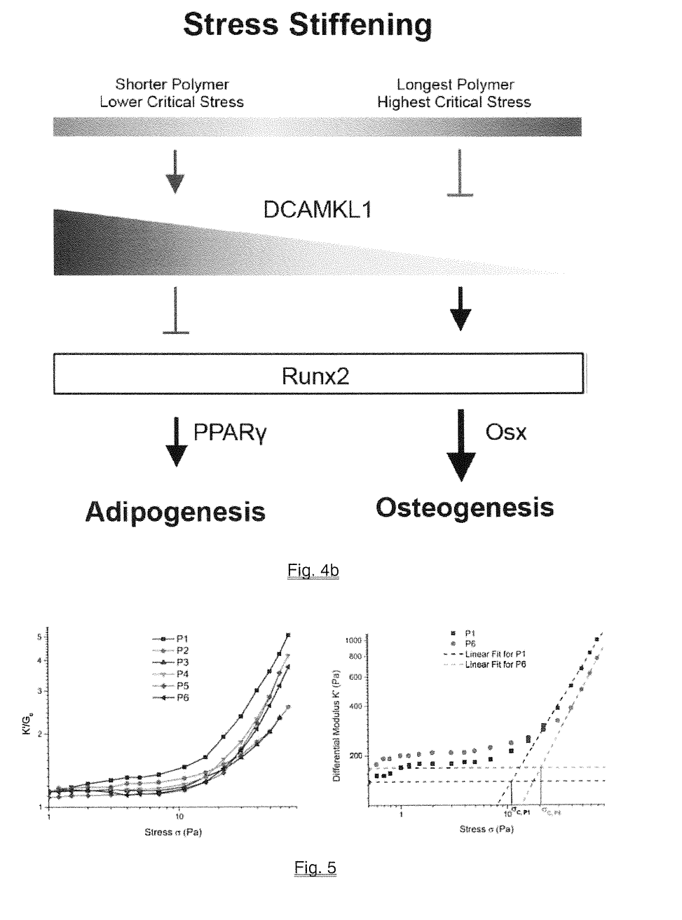

A recent report has indicated that the microtubule-associated protein DCAMKL1 represses RUNX2, an early osteogenesis marker, and thus regulates osteogenic differentiation in vitro and in an in vivo rat model. DCAMKL1 is also known to enhance microtubule polymerization. Furthermore, it has also been reported that microtubule de-polymerization can alter the myosin mechanochemical activity through myosin regulatory side chain phosphorylation, thus resulting in increased actomyosin contraction. We therefore investigated the role of DCAMKL1 in the stress-stiffening mediated control of hMSCs differentiation in our 3D culture system as a function of the gel critical stress. Interestingly, western blot analysis revealed a negligible DCAMKL1 expression for the polymer gel with the highest critical stress (P6) and a significant increase in the expression of this protein with decreasing the critical stress for stress-stiffening (FIG. 4a). Concomitantly, RUNX2 protein expression was not observed in the gels with lower critical stress (P1-P3) while the protein was clearly expressed in the higher critical stress polymers (P4-P6) in correlation with the observed osteogenic commitment in these gels. This is also in agreement with the observed overall increase in the RUNX2 mRNA expression between the shorter (P1-P3) and longer (P4-P6) polymers (FIG. 3f), although to a lesser extent, but still significant. These observations correlate well with preferential osteogenesis in gels of higher critical stress and lack of osteogenic commitment as the critical stress for stress-stiffening is lowered. A plot of the relative intensities (protein expression) of RUNX2 versus DCAMKL1 for all the conditions (P1 to P6) showed a switch-like relationship between these two proteins with the existence of a threshold value for the expression of DCAMKL1, which antagonizes RUNX2 in adipogenic lineage commitment (FIG. 4a). This observation has functional relevance for our mechanistic interpretations as it correlates with the observed stress-stiffening mediated commitment switch.

In order to further confirm the functional relationship between the two proteins in our stress-stiffening gel systems, DCAMKL1 gene silencing (through shRNA) and overexpression (via transient transfection) were performed for the hMSCs cultured in the P1 and P6 polymer gels. The DCAMKL1 silencing resulted in the increased expression of RUNX2 for the P1 polymer gel as well as for the P6 polymer gel but to a lesser extent. In contrast, DCAMKL1 overexpression did not significantly alter the expression of RUNX2 in the P1 polymer gel while a significant decrease was observed for P6. These data confirm the functional relationship between the two proteins in our gel system with DCAMKL1 being “upstream” of RUNX2 with a switch-like relationship, along with the existence of a threshold value for the expression of DCAMKL1, which inhibits the expression of RUNX2. In addition these data are in agreement with the previous in vivo and in vitro study.

Altogether these results are the first report of a microtubule-associated protein DCAMKL1 being involved in a new stress-stiffening mediated mechanotransduction pathway involving microtubule dynamics for the control of hMSCs differentiation (FIG. 4b). These data indicate that, stem cell fate is regulated by ECM stress stiffening via a different molecular mechanism than the one described for classical 2D substrate rigidity sensing.

Methods

Azide—Functionalized Polymer Synthesis (General Procedure). A solution of catalyst Ni(ClO₄)₂.6H₂O (1 mM) in toluene/ethanol (9:1) was added to a solution of non-functionalized monomer 1 and azide appended monomer 2 in freshly distilled toluene (50 mg/mL total concentration; molar ratio 1/2=100) in required amount and the reaction mixture was stirred at room temperature (20° C.) for 72 h. The resultant polymer was precipitated 3 times from dicholoromethane in di-isopropyl ether and dried overnight in air. The polymer was characterized by rheology, viscometry and AFM analysis.
Synthesis of P1′: The catalyst to monomer (1+2) molar ratio used: 1/1000
Synthesis of P2′: The catalyst to monomer (1+2) molar ratio used: 1/2500
Synthesis of P3′: The catalyst to monomer (1+2) molar ratio used: 1/3000
Synthesis of P4′: The catalyst to monomer (1+2) molar ratio used: 1/4000
Synthesis of P5′: The catalyst to monomer (1+2) molar ratio used: 1/6000
Synthesis of P6′: The catalyst to monomer (1+2) molar ratio used: 1/8000

Conjugation of Azide-Functionalized Polymers with GRGDS Peptide: The GRGDS peptide was dissolved in borate buffer (pH 8.4) at a concentration of 2 mg/mL. A solution of BCN-NHS in DMSO was added to the peptide solution in borate buffer in 1:1 molar ratio and stirred on roller-mixer for 3 h at room temperature (20° C.). The formation of BCN-GRGDS conjugate was confirmed by mass spectrometry. MS calc.: 910.4, obtained: 911.4

The azide functionalized polymer (P1′-P6′) was dissolved in acetonitrile at a concentration of 3 mg/mL. To this solution, the appropriate volume of BCN-GRGDS solution in borate buffer (based on the molar equivalent of azide functions of the polymer) was added. The mixture was allowed to stir on roller-mixer for 72 h at room temperature (20° C.). The resultant polymer-peptide conjugates (P1-P6) were precipitated by adding the reaction mixture drop wise to di-isopropyl ether.

Determination of the Amount of Azides on the Azide Functionalized Polymer:

A dichloromethane solution of BCN conjugated lissamine dye was added to a dichloromethane solution of the polymer (1 mg/mL) in 1:1.2 molar ratio w.r.t. the calculated amount of azides in an azide polymer. The reaction mixture was rotated at 15 rpm in dark for 12 h at room temperature (20° C.). The polymer-dye conjugate was precipitated 4 times from dichloromethane in di-isopropylether, dried in air overnight, re-dissolved in dichloromethane, after which the absorption spectra were recorded. The extinction coefficient of 138,428 Lmol⁻¹cm⁻¹ was used at a wavelength of 559 nm to determine the amount of dye attached to the polymer, and thus to calculate the amount of azide present on the polymer (Table 1).

Rheology Analysis: The polymers were dissolved at a concentration of 2 mg/mL in α-MEM (without serum) by gentle rotation (7-8 rpm) at 4° C. on a 90° rotor for 36 h. For determining the bulk stiffness of the gel, a variable temperature rheology was performed (plate-plate geometry; 250 μm geometry gap), by heating the solution from 5° C. to 37° C. at a heating rate of 2° C./min at a constant strain of 2% and constant frequency of 1 Hz. This experiment was immediately followed by a time sweep experiment (5 min.) at 37° C. at a constant frequency of 1 Hz and the G′ observed at the end of the experiment was taken as the equilibrium bulk stiffness of the gel at this temperature. For non-linear rheology, the previously described pre-stress protocol was employed immediately after the aforementioned time sweep experiment.

The critical stress σ_{c} was determined by the rheology analysis. For further details of determining the critical stress σ_{c}, Jaspers, M. et al. Ultra-responsive soft matter from strain-stiffening hydrogels. Nat. Commun. 5, 5808 (2014), FIG. 2 and the section titled Mechanical analysis (p2-3) and the section titled Rheology (p7), incorporated by reference, are referred.

The critical stress σ_{c} was determined by fitting (if possible) or by visual inspection of the obtained differential modulus (K′) as a function of stress. Fitting was performed by fitting the non-linear regime to a single exponent (K′=aσ^{m}) to calculate σ_{c} as the intercept between the fitted line and the region where K′ equals G₀. When not enough data points could be recorded, the onset of deviation of linearity is taken as the σ_{c}.

Atomic Force Microscopy: To visualize individual polymer chains and determine the average length of the polymers, solutions (˜1 μg/mL inCHCl₃) were spin coated (300 rpm for 20 seconds) on freshly cleaved mica substrates and imaged by using AFM tapping mode. Polymer lengths were determined by using the ImageJ software. The lengths of at least 150 polymer chains were counted to obtain the distribution and the mean of the polymer chain length for any particular sample.

Cell Culture. Human Mesenchymal Stem Cells (hMSCs) were obtained from Lonza, Inc. (Switzerland). Cells were then cultured in α-MEM medium (lnvitrogen) supplemented with 10% fetal bovine serum (FBS), 1% penicillin/streptomycin and incubated in a humidified atmosphere containing 5% (v/v) CO₂ at 37° C. For the encapsulation of cells in the gels, first, the cell pellets were obtained by centrifugation. Then 500 μL of the cold polymer solution (˜10° C.) was added directly to the pellet, followed by a gentle pipetting up and down 3-4 times to ensure a homogeneous mixture that was directly put onto a cover slip in a 6-well plate (also kept cold). Thereafter, the solution was sandwiched between two cover slips and the well plate was transferred to a 37° C. incubator. The volume of the suspension was chosen (500 μL) in order to obtain hydrogel thickness in the range of 3 mm. The polymer solution forms a gel immediately after incubation at 37° C. as revealed by kinetic rheology experiments. Afterwards, the gel becomes stiffer with time and attains the final stiffness in 2-3 minutes. This favors the supporting of cells in 3D rather than the cells settling at the bottom. After gel formation, the two cover slips were removed and α-MEM medium (without serum) was added. All cell culture experiments were carried out without any serum in the medium for the first 6 h of culture. Then, α-MEM medium with 10% serum was added. All cells were used at low passage numbers (≤passage 4), were subconfluently cultured and were seeded at 10⁶ cells/mL for the purpose of the experiments and in order to avoid cell-cell contacts. The lineage commitment and differentiation of the gel encapsulated hMSCs after 96 h and 3 weeks of culture, respectively, were investigated with bipotential differentiation medium (1:1 v/v osteogenic and adipogenic media, Lonza). For all the experiments, a non-functionalized soft polymer gel (cell culture in growth medium) served as control. The live/dead viability assay at 3 weeks in these control gels indicated excellent cell viability. The pharmacological agents used were 50 μM Blebbistatin (EMD Biosciences-Calbiochem), 1 μM cytochalasin D (Sigma) and 50 nM Taxol (Abcam). The hMSCs were exposed to each pharmacological agent for 1 h, 24 h and 72 h, respectively, after seeding on a modified polymer. For antibody inhibition studies, cells were preincubated with 5 ng/mL anti-α1, 2, 3 and 5-β1, 2 (all from Santa Cruz Biotechnology). For competition experiments with soluble RGD peptides, the cells were incubated in 1 mL of cell culture media containing 200 μg of RGDS peptides during 20 min on plastic and then transferred to the polymer gels. To evaluate proliferation, total double-stranded DNA content was determined by using the PicoGreen assay as previously reported.

Confocal Microscopy. In order to assess the homogeneous distribution of cells in our hydrogels, very thin slices of the gel were cut transversely at various depths, including the two interfaces. The fluorescently labeled cells encapsulated in the gel slices were imaged by confocal microscopy with a Leica SP5 confocal microscope, 10× objective, 0,3 NA. 400 μm thick z-stacks were then acquired every 2,39 μm and the 3D images were reconstructed by using the Imaris 7.0 software.

MTT Assay. As described in literature, briefly, cell viability was determined by the 3-(4,5-dimethylthiazol-2-yl)-2,5-diphenyltetrazolium bromide (MTT) assay and the data are presented as a percentage of control viability.

Live/Dead Staining. Cell viability was determined with the live/dead viability/cytotoxicity kit (Molecular Probes), according to the manufacturer's protocol.

Real-Time PCR Analysis of Gene Expression. RT-PCR was performed as previously described. Briefly, total RNA was extracted by using the RNeasy total RNA kit from Qiagen in accordance with the manufacturer's instructions. Purified total RNA was used to make cDNA by reverse transcription reaction (Gibco BRL) by using random primers (Invitrogen). Real-time PCR was performed by using SYBR green reagents (Bio-Rad). The data were analyzed by using the iCycler IQTM software. The cDNA samples (1 μL in a total volume of 20 μL) were analyzed for the gene of interest and for the house-keeping gene GAPDH. The comparison test of the cycle-threshold point was used to quantify the gene expression level in each sample. The primers used for the amplification are listed in Supplementary Table 1.

Western Blotting. After 96 h, the polymer gels were exposed to a cold environment (around 10° C.). The cell pellet was obtained by centrifugation. The cells were permeabilized (10% SDS, 25 mM NaCl, 10 nM pepstatin and 10 nM leupeptin in distilled water and loading buffer), boiled for 10 min and resolved by reducing PAGE (Invitrogen). Proteins were transferred onto nitrocellulose, blocked, and labeled with HRP-conjugated antibodies (Invitrogen). The microtubule associated protein DCAMKL1 was blotted by using the monoclonal anti-DCAMKL1 antibody (Santa Cruz Biotechnology). The transcriptional factor RUNX2 was blotted by using the monoclonal anti-Runx2 antibody (Abcam). The western blots in these experiments were run in triplicate, along with an additional blot for tubulin and Coomassie Blue staining to ensure consistent protein load between samples. In order to construct the plot of the relative intensities of RUNX2 versus DCAMKL1 (FIG. 4a) and to illustrate the switch-like relationship between the two proteins, the “zero” of the RUNX2 relative intensity was set at the corresponding level of expression of RUNX2 in hMSCs cultured on plastic, which was set to 1.

Immunostaining. After 96 h of culture, the gels were exposed to cold environment (˜10° C.), the cell pellet was collected from the fluid by centrifugation, transferred onto the well plate and allowed to adhere to the well plate surface by culturing in A-MEM with serum for 16 h. The cells were then fixed for 20 min in 4% paraformaldehyde/PBS at ˜37° C. After fixation, the cells were permeabilized in a PBS solution of 1% TritonX100 for 15 min. The cells were then incubated with primary antibody (mouse anti-vinculin for adhesion, mouse anti-STRO-1 for differentiation) for 1 h at 37° C. After washing, cells were stained with Alexa Fluor® 647 rabbit anti-mouse IgG secondary antibody for 30 min. at ˜37° C. Cell cytoskeletal filamentous actin (F-actin) was visualized by treating the cells with 5 U/mL Alexa Fluor® 488 Phalloidin (Sigma, France) for 1 h at 37° C. Vinculin was visualized by treating the cells with 1% (v/v) monoclonal anti-vinculin (clone hVIN-1 antibody produced in mouse) for 1 h at 37° C. The cells were then stained with Alexa fluor® 568 (F(ab′)2 fragment of rabbit anti-mouse IgG(H+L)) during 30 min at room temperature. After 96 h, Osterix was visualized by treating the cells with 1% (v/v) rabbit monoclonal anti-Osterix (antibody produced in rabbit) for 1 h at 37° C. The cells were then stained with Alexa fluor® 568 (F(ab′)2 fragment of mouse anti-rabbit IgG(H+L)) during 30 min at room temperature. Tubulin (stained by Anti-Tubulin β3 (Sigma, France) was visualized by treating the cells with 1% (v/v) monoclonal anti-Tubulin β3 (Abcam, Cambridge), for 1 h at 37° C. and then with Alexa Fluor® 588 (F(ab′)2 fragment of goat anti-rabbit IgG(H+L)) for 30 min at room temperature. There was no detection of the muscle transcription factor MyoD1 (stained with anti-MyoD1 (Santa Cruz Biotechnology, USA)). To stain lipid fat droplets, the cells were fixed in 4% paraformaldehyde, rinsed in PBS and 60% isopropanol, stained with 3 mg ml⁻¹ Oil Red O (Sigma, France) in 60% isopropanol and rinsed in PBS at ˜37° C.

For quantification of STRO-1, Osterix, Tubulin β3, MyoD1 and lipid fat droplets, positive contacts number and areas, we used the freeware image analysis ImageJ® software. First the raw image was converted to an 8-bit file, and the unsharp mask feature (settings 1:0.2) was used to remove the image background (rolling ball radius 10). After smoothing, the resulting image, which appears similar to the original photomicrograph but with minimal background, was then converted to a binary image by setting a threshold. The threshold values were determined by selecting a setting, which gave the most accurate binary image for a subset of randomly selected photomicrographs with varying glass substrates. The total contact area and mean contact area per cell were calculated by “analyse particules” in Image J. A minimum of 20 to 30 cells per condition were analyzed.

Statistical Analysis. In terms of fluorescence intensity, sub-cell contact area and real-time PCR assay, the data were expressed as the mean±standard error, and were analyzed by using the paired Student's t-test method. Significant differences were designated for P values of at least <0.01.

Overexpression of DCAMKL1. The overexpression of DCAMKL1 was performed as previously described by Lin PT et a1⁵⁷. Briefly, Human DCAMKL1 was cloned by RT-PCR using primers directed toward the human sequence and was subsequently sequenced. Full-length human DCAMKL1 was subsequently cloned into the Kpnl site of pcDNA3.1 C(−) (Invitrogen, Carlsbad, Calif.) and overexpressed by transient transfection with Super-fectamine (Qiagen, Chatsworth, Calif.) according to the manufacturer's recommendations. The efficiency of the DCAMKL1 overexpression was assessed by western blot for hMSCs cultured on plastic. A 180-200% increase in protein level was observed after 72 h.

DCAMKL1 shRNA Silencing. DCAMKL1 silencing has been performed by transfecting hMSCs with a pool of 3 target-specific lentiviral vector plasmids each encoding 19-25 nt (plus hairpin) shRNAs designed to knock down gene expression (Santa Cruz Biotechnology). A mock plasmid was transfected as a control. Transient transfection was performed by using Lipofectamine 2000 (Invitrogen) according to the manufacturer's protocol. The efficiency of the DCAMKL1 silencing was assessed by western blot for hMSCs cultured on plastic. The DCAMKL1 silencing decreased DCAMKL1 mRNA level by 50-60% (not shown) and DCAMKL1 protein level by 60-70% after 24 h.

TABLE 1
Properties of oligo(alkylene glycol) functionalized co-polyisocyanopeptide
P1-P6

| | | Viscosity | | Mean | Mean Critical |
|---|---|---|---|---|---|
| | | derived | Average | (GRGDS | Stress |
| | | molecular | spacing of | functionalized) | (σ_{c}, Pa) in α- |
| | | weight | —N3 on the | polymer | MEM gels at |
| | Catalyst/ | (N3-polymer; | polymer | length from | 2 mg/mL |
| Polymer | monomer | kg/mol) | chain (nm) | AFM (nm) | concentration |
| P1 | 1/1000 | 307 | 14 | 182 | 9.4 |
| P2 | 1/2500 | 426 | 14 | 226 | 9.9 |
| P3 | 1/3000 | 491 | 18 | 250 | 12.8 |
| P4 | 1/4000 | 571 | 15.6 | 309 | 14.6 |
| P5 | 1/6000 | 591 | 14 | 367 | 16.6 |
| P6 | 1/8000 | 685 | 17 | 434 | 19.3 |

It was observed that the use of polymers P1-P3 led to adipogenic differentiation whereas the use of polymers P4-P6 led to osteogenic differentiation.

Experiment 2

Oligo(alkyleneglycol)-substituted polyisocyanopeptides were prepared by using various ratios catalyst/monomer as shown in table 1. GRGDS was used as the cell adhesion factor. The decrease in the catalyst/monomer ratio resulted in an increase in viscosity average molecular weight (Mv) and the mean polymer length, while the distribution of the cell adhesion factor over the polymer chain remained at a constant level of 1 cell adhesion factor per 14-18 nm of polymer backbone. The relationship between the molecular weight and the mean polymer length can also be derived from table 1.

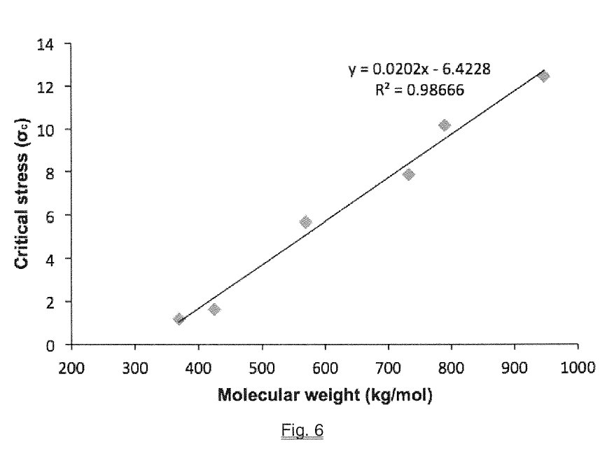

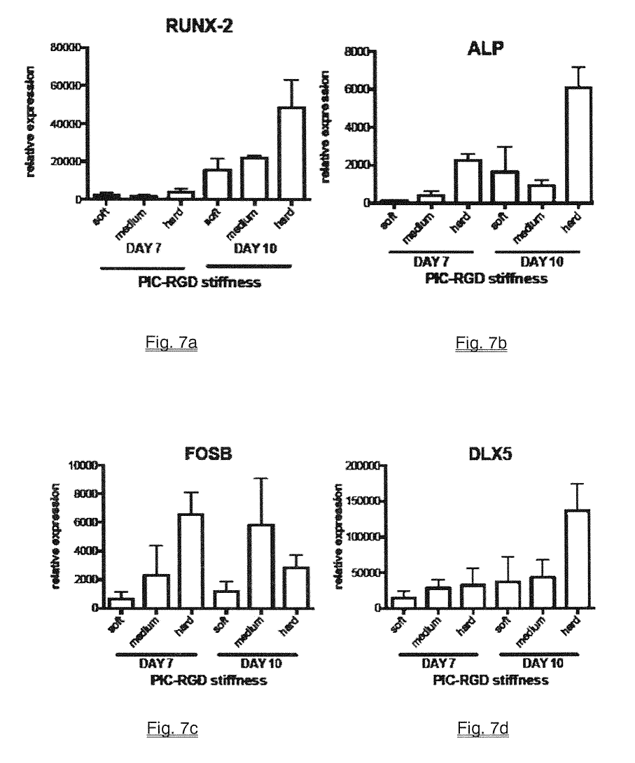

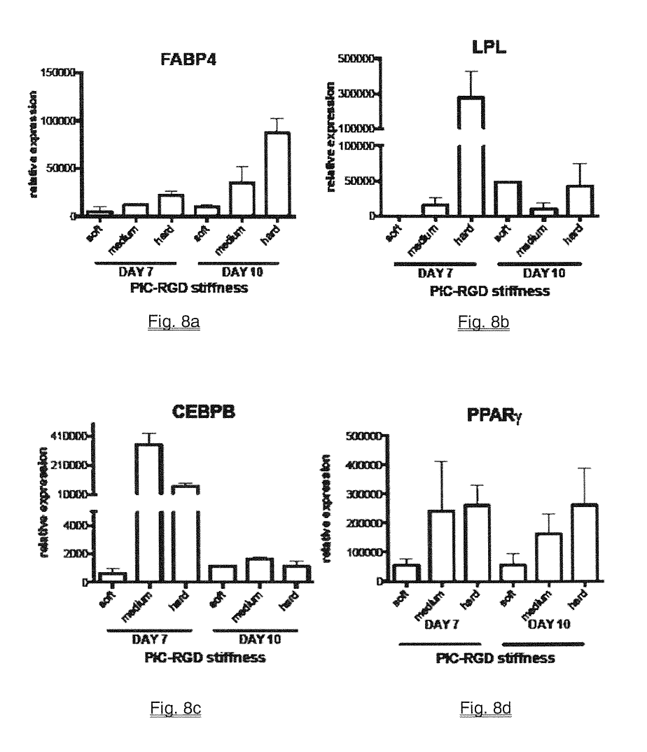

FIG. 6 shows the relationship between the molecular weight of the polyisocyanopeptides used according to the invention and the critical stress of the hydrogel made using the polyisocyanopeptides.

Experiment 3

Polymer Preparation

Polyisocyanopeptides (P7′-P9′) were synthesized as described above.

The catalyst to monomer molar ratio was 1:1000, 1:5000 and 1:7000 respectively, to obtain polymers of increasing molecular weight (determined by viscosity measurements, Table 2) (P7′-P9′). These azide functionalized polymers were then subjected to strain-promoted click reaction with BCN-RGD10 (BCN: Bicyclo[6.1.0]non-4-yn-9-ylmethyl) to obtain cell adhesive RGD10 functionalized polymers (PIC-RGD10) P7-P9. The strain-promoted click reaction is performed in the same way as described for functionalization with BCN-GRGDS under Methods above.

TABLE 3
Properties of oligo(alkylene glycol) functionalized co-polyisocyanopeptide
P7-P9

| | | | | G′ @37° C., | |
|---|---|---|---|---|---|
| Code | Polymer | σ_{c}, Pa | LOST, ° C. | Pa** | Mv, kDa |
| P7 | RGD10 1k | 7* | 18 | 78 | 375 |
| P8 | RGD10 5k | 18* | 15 | 230 | 545 |
| P9 | RGD10 7k | 23.6 | 14 | 214 | 614 |

*Plate slipping/Gel braking resulting in not enough data points for fitting to obtain σ_{c} decimals. Values obtained by visual inspection of the data.
**The G′ values are measured in incomplete α-MEM.

The average viscosity molecular weight, M, of the polymers was calculated using the empirical Mark-Houwink equation, [η]=KMᵥ^{a}, where [η] is the intrinsic viscosity of the polymer solution (in acetonitril) as determined from viscometry measurements, using a Ostwald tube, and Mark-Houwink parameters K and a depend on polymer and solvent characteristics. We used values that were previously determined for (other) rigid polyisocyanides: K=1.4×10−9 and a=1.75 (Van Beijnen, A., Nolte, R., Drenth, W., Hezemans, A. & Van de Coolwijk, P. Helical configuration of poly(iminomethylenes). Screw sense of polymers derived from optically active alkyl isocyanides. Macromolecules 13, 1386-1391 (1980).)

Effect of Stress-Stiffening on hASC Differentiation

Human adipose derived stem cells (hASCs, passage 3) were cultured in αMEM (Sigma, Germany) supplemented with 10% fetal calf serum (FCS) and 1% Penicilin/Streptomycin (P/S), until reaching 70% confluence. The cells were trypsinized and prepared in a suspension of 10⁶ cells in complete αMEM. Equal volumes of cells suspension and cold PIC-RGD10 solution, previously prepared at 4 mg/ml in complete αMEM, were slowly mixed until cells were evenly distributed within the gel, thus rendering a 2 mg/mL gel suspension containing 0.5×10̂6 cells/ml. Three different PIC-RGD10 batches with different stiffness (soft, intermediate, hard) were used for encapsulation of hASCs. 150 uL of the gel-hASCs suspension were carefully loaded into 48-well plates wells allowed to solidify at 37° C. After 10 minutes, 200 uL of warmed αMEM were gently added to each well and cultured overnight at 37° C. and 5% CO₂. The next day, used media was replenished with different media, depending on the experiment.

osteogenic differentiation medium (OST) consisting of complete αMEM, 50 mM β-glycerophosphate anhydrous, 50 μg/ml ascorbic acid and 10⁻⁸ M dexamethasone (results FIG. 7)

commercially available adipogenic differentiation medium (ADIPO, Stemcell technologies, Cat Nr. 05412) Results FIG. 8

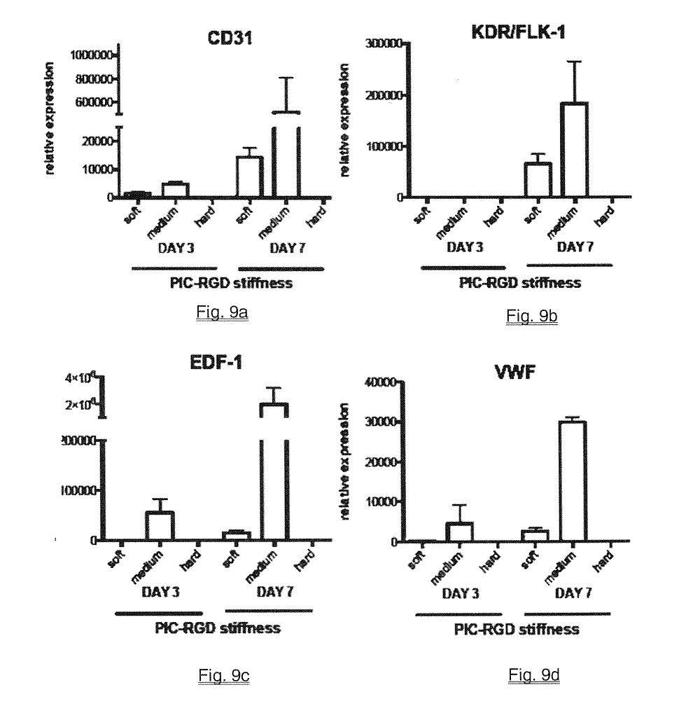

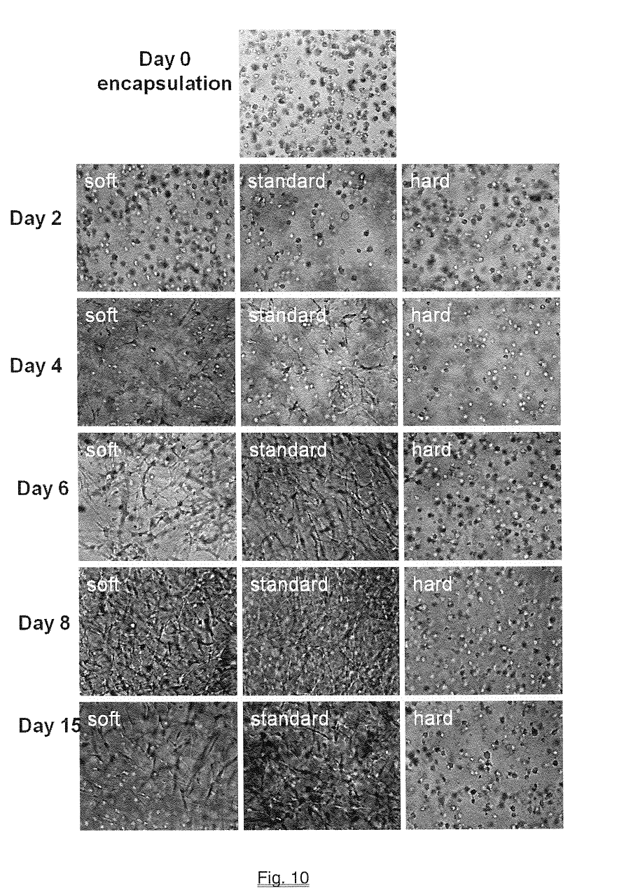

endothelial differentiation medium (ENDO) consisting of DMEM high glucose supplemented with 50 ng/mL recombinant vascular endothelial growth factor (rhVEGF) and 10 ng/mL recombinant basic fibroblast growth factor (rhbFGF), 2% FCS and 1% P/S (results FIG. 9)

complete αMEM (control medium) (results FIG. 10)

Cells were allowed to grow in the gels for 21 days with replenishment of media every 3 days. At days 3, 7, 14 and 21, samples were retrieved and stored in 800 μL TRIzol reagent (Life Technologies) for mRNA extraction and conversion to cDNA. Real time RT-PCR reactions were carried out for osteogenic (RUNX2, ALP, FOSB and DLX5), endothelial (EDF1, VWF, KDR/FLK-1, and CD31), adipogenic (PPARγ, CEBPB, LPL and FABP4) and sternness (STRO1, ENG, NT5E and THY-1)—specific genes.

The media composition triggers the differentiation, while material properties of the RGD10-functionalized polyisocyanopeptides (P7-P9) (such as stiffness, RGD10 content, etc) support and enhance certain differentiation pathways.

In FIG. 7a-d can be seen that osteogenic differentiation of the stem cells is primarily supported by hydrogels of polymer P9 with the highest viscosity and the highest critical stress.

According to FIG. 8a-d, the adipogenic differentiation of the stem cells is supported in the environment of hydrogels of polymers P8 and P9, with a medium to high viscosity and a medium to high critical stress.

FIG. 9a-d shows that the endothelial differentiation is supported primarily in the environment of hydrogels of polymer P8 with a medium viscosity and a medium value of the critical stress.

The cell morphology of the hASCs in a non-differentiating α-MEM cell growth medium combined with the three different PIC-RGD10 batches with different stiffness (soft, intermediate, hard) was studied for 15 days. FIG. 10 shows photographs of the hASCs in the hydrogels over time.

In FIG. 10 it can be observed that the cells grow fast in the soft hydrogel and slower in the intermediate (standard) hydrogel. Growth of the hASCs is shown by the stretched morphology of the cells. In the hard hydrogel the cells did not grow and remained round.

In experiment 3, the cells are grown in a single differentiation medium, while in experiment 1 the cells are grown in a bipolar medium, which gives the cells the opportunity to grow and differentiate in two directions: either adipogenic or osteogenic.

What is claimed is:

1. A cell culture comprising:
a) a cell culturing medium for growing stem cells,
b) a three-dimensional (3D) cell growth matrix and
c) stem cells,
wherein the cell culture has a critical stress σ_{c} of 2-30 Pa, wherein the critical stress is a stress which marks an onset of strain stiffening and
wherein the cell culture has a storage modulus G′ measured at 37° C. of 50-1000 Pa, preferably σ_{c} ranges between 5-25 Pa and G′ measured at 37° C. ranges between 70-400 Pa.

2. The cell culture according to claim 1, wherein the 3D cell growth matrix comprises at least one of Matrigel®, Puramatrix®, Raft® 3D, Insphero®, Bioactive 3D®, Cellusponge®, Optimaix® and GroCell-3D® scaffolds.

3. The cell culture according to claim 1, wherein the 3D cell growth matrix comprises an oligo(alkylene glycol) substituted co-polyisocyanopeptide.

4. The cell culture according to claim 3, wherein a concentration of the polyisocyanopeptide in the 3D cell growth matrix is 1-5 mg/ml.

5. The cell culture according to claim 3, wherein an average length of the polyisocyanopeptide is 250-680 nm as determined by AFM.

6. The cell culture according to claim 3, wherein the polyisocyanopeptide has a cell adhesion factor covalently bound to the polyisocyanopeptide and/or the cell culturing medium comprises fibrin,
wherein when the polyisocyanopeptide has a cell adhesion factor covalently bound to the polyisocyanopeptide, the average distance between the cell adhesion factors along the polyisocyanopeptide backbone is 10-50 nm.

7. A method for inducing differentiation of stem cells, comprising the steps of:
a) mixing a cell culturing medium for differentiation of stem cells with an oligo(alkylene glycol) substituted co-polyisocyanopeptide at a temperature between 0 and 18° C. to obtain a polymer solution;
b) mixing the polymer solution with stem cells at a temperature between 0 and 18° C. to obtain a cell culture solution;
c) allowing the cell culture solution to warm to a temperature between 30 and 38° C. to form a cell culture comprising a hydrogel and allow the stem cells to differentiate,
wherein a concentration of the polyisocyanopeptide in the polymer solution is 1-5 mg/ml,
wherein an average length of the polyisocyanopeptide is 250-680 nm as determined by AFM,
wherein a cell density of the stem cells in the cell culture solution is 0.3*10⁶-1*10⁶ cells/ml,
wherein the hydrogel has a critical stress σ_{c} of 2-30 Pa, wherein the critical stress is a stress which marks an onset of strain stiffening,
wherein the hydrogel has a storage modulus G′ measured at 37° C. of 50-1000 Pa and
wherein the polyisocyanopeptide has a cell adhesion factor covalently bound to the polyisocyanopeptide and/or wherein the cell culturing medium comprises fibrin,
wherein when the polyisocyanopeptide has a cell adhesion factor covalently bound to the polyisocyanopeptide, the average distance between the cell adhesion factors along the polyisocyanopeptide backbone is 10-50 nm.

8. A method for inducing osteogenic differentiation of stem cells, comprising the steps of:
a) mixing a cell culturing medium for osteogenic differentiation with an oligo(alkylene glycol) substituted co-polyisocyanopeptide at a temperature between 0 and 18° C. to obtain a polymer solution;
b) mixing the polymer solution with stem cells at a temperature between 0 and 18° C. to obtain a cell culture solution;
c) allowing the cell culture solution to warm to a temperature between 30 and 38° C. to form a cell culture comprising a hydrogel and allow the stem cells to differentiate,
wherein a concentration of the polyisocyanopeptide in the polymer solution is 1-5 mg/ml,
wherein an average length of the polyisocyanopeptide is 250-680 nm as determined by AFM,
wherein a cell density of the stem cells in the cell culture solution is 0.3*10⁶-1*10⁶ cells/ml,
wherein the hydrogel has a critical stress σ_{c} of 13-30 Pa, wherein the critical stress is a stress which marks an onset of strain stiffening,
wherein the hydrogel has a storage modulus G′ measured at 37° C. of 50-1000 Pa, preferably between 70-350 Pa, more preferably between 72-300 Pa, and
wherein the polyisocyanopeptide has a cell adhesion factor covalently bound to the polyisocyanopeptide and/or wherein the cell culturing medium comprises fibrin,
wherein when the polyisocyanopeptide has a cell adhesion factor covalently bound to the polyisocyanopeptide, the average distance between the cell adhesion factors along the polyisocyanopeptide backbone is 10-50 nm.

9. A method for inducing vascularization of stem cells, comprising the steps of:
a) mixing a cell culturing medium for vascularization with an oligo(alkylene glycol) substituted co-polyisocyanopeptide at a temperature between 0 and 18° C. to obtain a polymer solution;
b) mixing the polymer solution with stem cells at a temperature between 0 and 18° C. to obtain a cell culture solution;
c) allowing the cell culture solution to warm to a temperature between 30 and 38° C. to form a cell culture comprising a hydrogel and allow the stem cells to differentiate,
wherein a concentration of the polyisocyanopeptide in the polymer solution is 1-5 mg/ml,
wherein an average length of the polyisocyanopeptide is 50-750 nm as determined by AFM,
wherein a cell density of the stem cells in the cell culture solution is 0.3*10⁶-1*10⁶ cells/ml,
wherein the hydrogel has a critical stress σ_{c} of 2-12 Pa, preferably 7-12 Pa, wherein the critical stress is a stress which marks an onset of strain stiffening,
wherein the hydrogel has a storage modulus G′ measured at 37° C. of 50-1000 Pa, preferably between 70-350 Pa, more preferably between 72-300 Pa and
wherein the polyisocyanopeptide has a cell adhesion factor covalently bound to the polyisocyanopeptide and/or wherein the cell culturing medium comprises fibrin,
wherein when the polyisocyanopeptide has a cell adhesion factor covalently bound to the polyisocyanopeptide, the average distance between the cell adhesion factors along the polyisocyanopeptide backbone is 10-50 nm.

10. A method for inducing adipogenic differentiation of stem cells, comprising the steps of:
a) mixing a cell culturing medium for adipogenic differentiation with an oligo(alkylene glycol) substituted co-polyisocyanopeptide at a temperature between 0 and 18° C. to obtain a polymer solution;
b) mixing the polymer solution with stem cells at a temperature between 0 and 18° C. to obtain a cell culture solution;
c) allowing the cell culture solution to warm to a temperature between 30 and 38° C. to form a cell culture comprising a hydrogel and allow the stem cells to differentiate,
wherein a concentration of the polyisocyanopeptide in the polymer solution is 1-5 mg/ml,
wherein an average length of the polyisocyanopeptide is 50-750 nm as determined by AFM,
wherein a cell density of the stem cells in the cell culture solution is 0.3*10⁶-1*10⁶ cells/ml,
wherein the hydrogel has a critical stress σ_{c} of 2-30 Pa, preferably 7-23 Pa or 8-20 Pa, wherein the critical stress is a stress which marks an onset of strain stiffening,
wherein the hydrogel has a storage modulus G′ measured at 37° C. of 50-1000 Pa, preferably between 70-350 Pa, more preferably between 72-300 Pa, and
wherein the polyisocyanopeptide has a cell adhesion factor covalently bound to the polyisocyanopeptide and/or wherein the cell culturing medium comprises fibrin,
wherein when the polyisocyanopeptide has a cell adhesion factor covalently bound to the polyisocyanopeptide, the average distance between the cell adhesion factors along the polyisocyanopeptide backbone is 10-50 nm, wherein the storage modulus G′ measured at 37° C. is 200-400 Pa and the viscosity average molecular weight (Mv) of the polyisocyanopeptide is between 100 and 1000 kg/mol, preferably 500-1000 kg/mol.

11. The method according to claim 8, wherein the storage modulus G′ measured at 37° C. is 200-400 Pa and the viscosity average molecular weight (Mv) of the polyisocyanopeptide is between 100 and 1000 kg/mol, preferably 200-700 kg/mol or 300-600 kg/mol.

12. The method according to claim 9, wherein the storage modulus G′ measured at 37° C. is 70-300 Pa and the viscosity average molecular weight (Mv) of the polyisocyanopeptide is between 100 and 1000 kg/mol, preferably 200-700 kg/mol or 300-600 kg/mol.

13. The method according to claim 10, wherein the storage modulus G′ measured at 37° C. is 70-450 Pa and the viscosity average molecular weight (Mv) of the polyisocyanopeptide is between 100 and 1000 kg/mol, preferably 200-700 kg/mol or 300-600 kg/mol.

14. The method according to claim 7, wherein the concentration of the polyisocyanopeptide in the polymer solution is 1.5-3 mg/ml.

15. The method according to claim 7, wherein the cell adhesion factor is covalently bound to the polyisocyanopeptide and wherein the polyisocyanopeptide is prepared by copolymerizing
i) a first comonomer of an oligo(alkylene glycol) functionalized isocyanopeptide grafted with a linking group and a second comonomer of a non-grafted oligo(alkylene glycol) functionalized isocyanopeptide,
wherein the molar ratio between the first comonomer and the second comonomer is 1:500 and 1:30; and
ii) adding a reactant of a spacer unit and a cell adhesion factor to the copolymer obtained by step a), wherein the spacer unit is represented by general formula A-L-B;
wherein the linking group and group A are chosen to react and form a first coupling and the cell adhesion factor and group B are chosen to react and form a second coupling,
wherein the first coupling and the second coupling are independently selected from the group consisting of alkyne-azide coupling, dibenzocyclooctyne-azide coupling, oxanorbornadiene-based-azide couplings, vinylsulphone-thiol coupling, maleimide-thiol coupling, methyl methacrylate-thiol coupling, ether coupling, thioether coupling, biotin-strepavidin coupling, amine-carboxylic acid resulting in amides linkages, alcohol-carboxylic acid coupling resulting in esters linkages and NHS-Ester (N-Hydroxysuccinimide ester)-amine coupling and
wherein group L is a linear chain segment having 10-60 bonds between atoms selected from C, N, O and S in the main chain.

16. The method according to claim 7, wherein the stem cells are chosen from human adipose stem cells and human mesenchymal stem cells.

17. The method according to claim 7, wherein the oligo(alkylene glycol) functionalized co-polyisocyanopeptide comprises a cell adhesion factor which is chosen from the group consisting of a sequence of amino acids of RGD, GRGDS, rhrVEGF-164 and rhrbFGF.

18. The cell culture according to claim 1, wherein the stem cells are chosen from human adipose stem cells and human mesenchymal stem cells.

19. The cell culture according to claim 3, wherein the oligo(alkylene glycol) functionalized co-polyisocyanopeptide comprises a cell adhesion factor which is chosen from the group consisting of a sequence of amino acids of RGD, GRGDS, rhrVEGF-164 and rhrbFGF.

20. (canceled)

21. (canceled)

22. (canceled)
